(19) United States
(12) Patent Application Publication
(10) Pub. No.: US 2002/0137054 A1
(43) Pub. Date: Sep. 26, 2002

(54) AXOR35, a G-protein coupled receptor

(75) Inventors: Kelly M. Aubart, Malvern, PA (US); Derk J. Bergsma, Berwyn, PA (US); Laura Fitzgerald, Kennett Square, PA (US); Todd L. Graybill, Pottstown, PA (US); Xiaotong Li, Devon, PA (US); David Michalovich, London (GB); Dwight M. Morrow, West Chester, PA (US); Yuan Zhu, Blue Bell, PA (US)

Correspondence Address: GLAXOSMITHKLINE, Corporate Intellectual Property - UW2220, P.O. Box 1539, King of Prussia, PA, 19406-0939, US

(73) Assignee: SmithKline Beecham Corporation & SmithKline Beecham plc

(21) Appl. No.: 09/910,411

(22) Filed: Jul. 20, 2001

Related U.S. Application Data
(63) Continuation of application No. 09/693,761, filed on Oct. 20, 2000, now abandoned.
Application No. 09/693,761 is a continuation-in-part of application No. 09/497,790, filed on Feb. 3, 2000, now abandoned.
Application No. 09/497,790 is a continuation-in-part of application No. 09/431,898, filed on Nov. 2, 1999, now abandoned.

(51) Int. Cl. C12Q 1/68; C07H 21/04; C07K 14/705; C12P 21/02; C12N 5/06

(52) U.S. Cl. 435/6; 435/69.1; 435/325; 435/320.1; 530/350; 536/23.2

(57) ABSTRACT

AXOR35 polypeptides and polynucleotides and methods for producing such polypeptides by recombinant techniques are disclosed. Also disclosed are methods for utilizing AXOR35 polypeptides and polynucleotides in diagnostic assays.

### REFERENCE TO RELATED APPLICATION

This is a continuation-in-part application of U.S. application Ser. No. 09/497,790 filed Feb. 3, 2000 which is also a continuation-in-part application of Ser. No. 09/431,898 filed Nov. 2, 1999, both applications are incorporated herein in their entirety.

### FIELD OF THE INVENTION

This invention relates to newly identified polypeptides and polynucleotides encoding such polypeptides, to their use in diagnosis and in identifying compounds that may be agonists, antagonists that are potentially useful in therapy, and to production of such polypeptides and polynucleotides.

### BACKGROUND OF THE INVENTION

The drug discovery process is currently undergoing a fundamental revolution as it embraces “functional genomics,” that is, high throughput genome- or gene-based biology. This approach as a means to identify genes and gene products as therapeutic targets is rapidly superseding earlier approaches based on “positional cloning.” A phenotype, that is a biological function or genetic disease, would be identified and this would then be tracked back to the responsible gene, based on its genetic map position.

Functional genomics relies heavily on high-throughput DNA sequencing technologies and the various tools of bioinformatics to identify gene sequences of potential interest from the many molecular biology databases now available. There is a continuing need to identify and characterize further genes and their related polypeptides/proteins, as targets for drug discovery.

It is well established that many medically significant biological processes are mediated by proteins participating in signal transduction pathways that involve G-proteins and/or second messengers, e.g., cAMP (Lefkowitz, Nature, 1991, 351:353-354). Herein these proteins are referred to as proteins participating in pathways with G-proteins or PPG proteins. Some examples of these proteins include the GPC receptors, such as those for adrenergic agents and dopamine (Kobilka, B. K., et al., Proc. Natl Acad. Sci., USA, 1987, 84:46-50; Kobilka, B. K., et al., Science, 1987, 238:650-656; Bunzow, J. R., et al., Nature, 1988, 336:783-787), G-proteins themselves, effector proteins, e.g., phospholipase C, adenyl cyclase, and phosphodiesterase, and actuator proteins, e.g., protein kinase A and protein kinase C (Simon, M. I., et al., Science, 1991, 252:802-8).

For example, in one form of signal transduction, the effect of hormone binding is activation of the enzyme, adenylate cyclase, inside the cell. Enzyme activation by hormones is dependent on the presence of the nucleotide GTP. GTP also influences hormone binding. A G-protein connects the hormone receptor to adenylate cyclase. G-protein was shown to exchange GTP for bound GDP when activated by a hormone receptor. The GTP-carrying form then binds to activated adenylate cyclase. Hydrolysis of GTP to GDP, catalyzed by the G-protein itself, returns the G-protein to its basal, inactive form. Thus, the G-protein serves a dual role, as an intermediate that relays the signal from receptor to effector, and as a clock that controls the duration of the signal.

The membrane protein gene superfamily of G-protein coupled receptors has been characterized as having seven putative transmembrane domains. The domains are believed to represent transmembrane α-helices connected by extracellular or cytoplasmic loops. G-protein coupled receptors include a wide range of biologically active receptors, such as hormone, viral, growth factor and neuroreceptors.

G-protein coupled receptors (otherwise known as 7TM receptors) have been characterized as including these seven conserved hydrophobic stretches of about 20 to 30 amino acids, connecting at least eight divergent hydrophilic loops. The G-protein family of coupled receptors includes dopamine receptors which bind to neuroleptic drugs used for treating psychotic and neurological disorders. Other examples of members of this family include, but are not limited to, calcitonin, adrenergic, endothelin, cAMP, adenosine, muscarinic, acetylcholine, serotonin, histamine, thrombin, kinin, follicle stimulating hormone, opsins, endothelial differentiation gene-1, rhodopsins, odorant, and cytomegalovirus receptors. Most G-protein coupled receptors have single conserved cysteine residues in each of the first two extracellular loops which form disulfide bonds that are believed to stabilize functional protein structure. The 7 transmembrane regions are designated as TM1, TM2, TM3, TM4, TM5, TM6, and TM7. TM3 has been implicated in signal transduction.

Phosphorylation and lipidation (palmitylation or farnesylation) of cysteine residues can influence signal transduction of some G-protein coupled receptors. Most G-protein coupled receptors contain potential phosphorylation sites within the third cytoplasmic loop and/or the carboxy terminus. For several G-protein coupled receptors, such as the β-adrenoreceptor, phosphorylation by protein kinase A and/or specific receptor kinases mediates receptor desensitization.

For some receptors, the ligand binding sites of G-protein coupled receptors are believed to comprise hydrophilic sockets formed by several G-protein coupled receptor transmembrane domains, said sockets being surrounded by hydrophobic residues of the G-protein coupled receptors. The hydrophilic side of each G-protein coupled receptor transmembrane helix is postulated to face inward and form a polar ligand binding site. TM3 has been implicated in several G-protein coupled receptors as having a ligand binding site, such as the TM3 aspartate residue. TM5 serines, a TM6 asparagine and TM6 or TM7 phenylalanines or tyrosines are also implicated in ligand binding.

G-protein coupled receptors can be intracellularly coupled by heterotrimeric G-proteins to various intracellular enzymes, ion channels and transporters (see, Johnson et al., Endoc. Rev., 1989, 10:317-331). Different G-protein α-subunits preferentially stimulate particular effectors to modulate various biological functions in a cell. Phosphorylation of cytoplasmic residues of G-protein coupled receptors has been identified as an important mechanism for the regulation of G-protein coupling of some G-protein coupled receptors. G-protein coupled receptors are found in numerous sites within a mammalian host.

Over the past 15 years, nearly 350 therapeutic agents targeting 7 transmembrane (7 TM) receptors have been successfully introduced onto the market.

### SUMMARY OF THE INVENTION

The present invention relates to AXOR35, in particular AXOR35 polypeptides and AXOR35 polynucleotides, recombinant materials and methods for their production. Such polypeptides and polynucleotides are of interest in relation to methods of treatment of certain diseases, including, but not limited to bacterial, fungal, protozoan and viral infections, particularly infections caused by HIV-1 or HIV-2; immunodeficiency; transplant rejection; gastrointestinal disorders such as gastric or duodenal ulcer, diarrhea, inflammatory bowel diseases such as Crohn's disease and ulcerative colitis; irritable bowel syndrome; vomiting; inflammation such as pruritis and atopic dermatitis; allergies and allergic disorders including asthma; allergic rhinitis; autoimmune disorders including, but not limited to: delayed type hypersensitivity; rheumatoid arthritis (RA), psoriasis, insulin dependent (Type I) diabetes (IDDM), multiple sclerosis, and scleroderma; urological diseases such as urinary retention, urinary incontinence, interstitial cystitis and benign prostatic hypertrophy; cardiovascular diseases such as angina pectoris, myocardial infarction, acute heart failure, myocardial ischemia, congestive heart failure; hypotension; hypertension; pulmonary disorders such as chronic obstructive pulmonary disease; cough; renal diseases such as renal ischemia, acute renal failure, and chronic renal failure; arteriosclerosis; arthrosclerosis; psychotic and neurological disorders, including migraine, chronic pain, bulimia, anorexia, anxiety, schizophrenia, manic depression, depression, delirium, dementia, and severe mental retardation; and dyskinesias, such as Parkinson's disease; Huntington's disease or Giles de la Tourette's syndrome; cancers including leukemias and lymphomas as well as other diseases such as type II diabetes, obesity, stroke, septic shock, graft versus host disease and osteoporosis, hereinafter referred to as “diseases of the invention”. In a further aspect, the invention relates to methods for identifying agonists and antagonists (e.g., inhibitors) using the materials provided by the invention, and treating conditions associated with AXOR35 imbalance with the identified compounds. In a still further aspect, the invention relates to diagnostic assays for detecting diseases associated with inappropriate AXOR35 activity or levels.

In accordance with another aspect of the present invention there are provided methods of screening for compounds which bind to and activate the AXOR35 polypeptides (receptors) of the present invention (called agonists), or inhibit the interaction of the AXOR35 polypeptides with receptor ligands (called antagonists). In particular, a preferred method for identifying agonist or antagonist of a receptor of the present invention comprises:

(a) contacting a cell expressing on the surface thereof the receptor, said receptor being associated with a second component capable of providing a detectable signal in response to the binding of a compound to said receptor, with a compound to be screened under conditions to permit binding to the receptor; and

(b) determining whether the compound binds to and activates or inhibits the receptor by measuring the level of a signal generated from the interaction of the compound with the receptor.

Further, preferred method for identifying agonist or antagonist of a receptor of the present invention comprises:

(a) contacting a cell expressing on the surface thereof the receptor, said receptor being associated with a second component capable of providing a detectable signal in response to the binding of a compound to said receptor, with a compound to be screened under conditions to permit binding to the receptor; and

(b) determining whether the compound binds to and activates or inhibits the receptor by comparing the level of a signal generated from the interaction of the compound with the receptor with the level of a signal without the presence of the compound.

In further preferred embodiments, the two methods described above further comprises conducting the identification of agonist or antagonist in the presence of labeled or unlabeled histamine or a histamine-like compound.

Thus, in another embodiment of the method for identifying agonist or antagonist of a receptor of the present invention comprises:

determining the inhibition of binding of a ligand to cells which have the receptor on the surface thereof, or to cell membranes containing the receptor, in the presence of a candidate compound under conditions to permit binding to the receptor, and determining the amount of ligand bound to the receptor, such that a compound capable of causing reduction of binding of a ligand is an agonist or antagonist. Preferably the ligand is histamine or a histamine-like compound. Yet more preferably histamine or a histamine-like compound is labeled. More particularly, a method of screening for AXOR35 receptor antagonist or agonist comprises the steps of:

(a) incubating a labeled histamine or a histamine-like compound with a whole cell expressing AXOR35 receptor on the cell surface, or cell membrane containing AXOR35 receptor;

(b) measuring the amount of labeled histamine or a histamine-like compound bound to the whole cell or the cell membrane;

(c) adding a candidate compound to a mixture of labeled histamine or a histamine-like compound and the whole cell or the cell membrane of step (a) and allowing to attain equilibrium;

(d) measuring the amount of labeled histamine or a histamine-like compound bound to the whole cell or the cell membrane after step (c); and

(e) comparing the difference in the labeled histamine or a histamine-like compound bound in step (b) and (d), such that the compound which causes the reduction in binding in step (d) is an agonist or antagonist.

In still another aspect, the invention relates to agonists and antagonists discovered by any of the present screening methods. Yet in still another aspect, the invention relates to treating conditions associated with AXOR35 imbalance with agonists or antagonists of AXOR35, in particular agonists and antagonists identified by any of the present screening methods.

Further, the present invention relates to a method of treating a disease of the invention, in particular asthma, by administering to a patient in need thereof an agonist or an antagonist of AXOR35. The present invention also relates to a method of agonizing or antagonizing AXOR35 for the treatment of a disease of the invention, in particular asthma, by administering to a patient in need thereof an agonist or an antagonist of AXOR35.

Yet in further aspect, the present invention relates to a method of treating a disease of the invention, in particular asthma, by administering to a patient in need thereof an agonist or an antagonist of AXOR35 identified by any of the herein described screening methods. Further the present invention also relates to a method of agonizing or antagonizing AXOR35 for the treatment of a disease of the invention, in particular asthma, by administering to a patient in need thereof an agonist or antagonist of AXOR35 identified by any of the herein described screening methods.

### DESCRIPTION OF THE INVENTION

In a first aspect, the present invention relates to AXOR35 polypeptides. Such polypeptides include:

(a) an isolated polypeptide encoded by a polynucleotide comprising the sequence of SEQ ID NO:1;

(b) an isolated polypeptide comprising a polypeptide sequence having at least 95%, 96%, 97%, 98%, or 99% identity to the polypeptide sequence of SEQ ID NO:2;

(c) an isolated polypeptide comprising the polypeptide sequence of SEQ ID NO:2;

(d) an isolated polypeptide having at least 95%, 96%, 97%, 98%, or 99% identity to the polypeptide sequence of SEQ ID NO:2;

(e) the polypeptide sequence of SEQ ID NO:2; and

(f) an isolated polypeptide having or comprising a polypeptide sequence that has an Identity Index of 0.95, 0.96, 0.97, 0.98, or 0.99 compared to the polypeptide sequence of SEQ ID NO:2;

(g) fragments and variants of such polypeptides in (a) to (f).

Polypeptides of the present invention are believed to be members of the 7TM receptor family of polypeptides. They are therefore of interest because G-protein coupled (7TM) receptors, more than other gene family, are targets of pharmaceutical intervention.

The biological properties of the AXOR35 are hereinafter referred to as “biological activity of AXOR35” or “AXOR35 activity.” Preferably, a polypeptide of the present invention exhibits at least one biological activity of AXOR35.

Polypeptides of the present invention also include variants of the aforementioned polypeptides, including all allelic forms and splice variants. Such polypeptides vary from the reference polypeptide by insertions, deletions, and substitutions that may be conservative or non-conservative, or any combination thereof. Particularly preferred variants are those in which several, for instance from 50 to 30, from 30 to 20, from 20 to 10, from 10 to 5, from 5 to 3, from 3 to 2, from 2 to 1 or 1 amino acids are inserted, substituted, or deleted, in any combination.

Preferred fragments of polypeptides of the present invention include an isolated polypeptide comprising an amino acid sequence having at least 30, 50 or 100 contiguous amino acids from the amino acid sequence of SEQ ID NO:2, or an isolated polypeptide comprising an amino acid sequence having at least 30, 50 or 100 contiguous amino acids truncated or deleted from the amino acid sequence of SEQ ID NO:2. Preferred fragments are biologically active fragments that mediate the biological activity of AXOR35, including those with a similar activity or an improved activity, or with a decreased undesirable activity. Also preferred are those fragments that are antigenic or immunogenic in an animal, especially in a human.

Fragments of the polypeptides of the invention may be employed for producing the corresponding full-length polypeptide by peptide synthesis; therefore, these variants may be employed as intermediates for producing the full-length polypeptides of the invention. The polypeptides of the present invention may be in the form of the “mature” protein or may be a part of a larger protein such as a precursor or a fusion protein. It is often advantageous to include an additional amino acid sequence that contains secretory or leader sequences, pro-sequences, sequences that aid in purification, for instance multiple histidine residues, or an additional sequence for stability during recombinant production.

Polypeptides of the present invention can be prepared in any suitable manner, for instance by isolation form naturally occurring sources, from genetically engineered host cells comprising expression systems (vide infra) or by chemical synthesis, using for instance automated peptide synthesizers, or a combination of such methods. Means for preparing such polypeptides are well understood in the art.

In a further aspect, the present invention relates to AXOR35 polynucleotides. Such polynucleotides include:

(a) an isolated polynucleotide comprising a polynucleotide sequence having at least 95%, 96%, 97%, 98%, or 99% identity to the polynucleotide sequence of SEQ ID NO:1;

(b) an isolated polynucleotide comprising the polynucleotide of SEQ ID NO:1;

(c) an isolated polynucleotide having at least 95%, 96%, 97%, 98%, or 99% identity to the polynucleotide of SEQ ID NO:1;

(d) the isolated polynucleotide of SEQ ID NO:1;

(e) an isolated polynucleotide comprising a polynucleotide sequence encoding a polypeptide sequence having at least 95%, 96%, 97%, 98%, or 99% identity to the polypeptide sequence of SEQ ID NO:2;

(f) an isolated polynucleotide comprising a polynucleotide sequence encoding the polypeptide of SEQ ID NO:2;

(g) an isolated polynucleotide having a polynucleotide sequence encoding a polypeptide sequence having at least 95%, 96%, 97%, 98%, or 99% identity to the polypeptide sequence of SEQ ID NO:2;

(h) an isolated polynucleotide encoding the polypeptide of SEQ ID NO:2;

(i) an isolated polynucleotide having or comprising a polynucleotide sequence that has an Identity Index of 0.95, 0.96, 0.97, 0.98, or 0.99 compared to the polynucleotide sequence of SEQ ID NO:1;

(j) an isolated polynucleotide having or comprising a polynucleotide sequence encoding a polypeptide sequence that has an Identity Index of 0.95, 0.96, 0.97, 0.98, or 0.99 compared to the polypeptide sequence of SEQ ID NO:2; and polynucleotides that are fragments and variants of the above mentioned polynucleotides or that are complementary to above mentioned polynucleotides, over the entire length thereof.

Preferred fragments of polynucleotides of the present invention include an isolated polynucleotide comprising an nucleotide sequence having at least 15, 30, 50 or 100 contiguous nucleotides from the sequence of SEQ ID NO:1, or an isolated polynucleotide comprising an sequence having at least 30, 50 or 100 contiguous nucleotides truncated or deleted from the sequence of SEQ ID NO:1.

Preferred variants of polynucleotides of the present invention include splice variants, allelic variants, and polymorphisms, including polynucleotides having one or more single nucleotide polymorphisms (SNPs).

Polynucleotides of the present invention also include polynucleotides encoding polypeptide variants that comprise the amino acid sequence of SEQ ID NO:2 and in which several, for instance from 50 to 30, from 30 to 20, from 20 to 10, from 10 to 5, from 5 to 3 from 3 to 2, from 2 to 1 or 1 amino acid residues are substituted, deleted or added, in any combination.

In a further aspect, the present invention provides polynucleotides that are RNA transcripts of the DNA sequences of the present invention. Accordingly, there is provided an RNA polynucleotide that:

(a) comprises an RNA transcript of the DNA sequence encoding the polypeptide of SEQ ID NO:2;

(b) is the RNA transcript of the DNA sequence encoding the polypeptide of SEQ ID NO:2;

(c) comprises an RNA transcript of the DNA sequence of SEQ ID NO:1; or

(d) is the RNA transcript of the DNA sequence of SEQ ID NO:1; and RNA polynucleotides that are complementary thereto.

The polynucleotide sequence of SEQ ID NO:1 shows homology with human histamine H3 receptor (Lovenberg, T. W. et al. Mol. Pharm. 55:1101-1107, 1999). The polynucleotide sequence of SEQ ID NO:1 is a cDNA sequence that encodes the polypeptide of SEQ ID NO:2. The polynucleotide sequence encoding the polypeptide of SEQ ID NO:2 may be identical to the polypeptide encoding sequence of SEQ ID NO:1 or it may be a sequence other than SEQ ID NO:1, which, as a result of the redundancy (degeneracy) of the genetic code, also encodes the polypeptide of SEQ ID NO:2. The polypeptide of SEQ ID NO:2 is related to other proteins of the 7TM receptor family, having homology and/or structural similarity with human histamine H3 receptor (Lovenberg, T. W. et al. Mol. Pharm. 55:1101-1107, 1999).

Preferred polypeptides and polynucleotides of the present invention are expected to have, inter alia, similar biological functions/properties to their homologous polypeptides and polynucleotides. Furthermore, preferred polypeptides and polynucleotides of the present invention have at least one AXOR35 activity.

Polynucleotides of the present invention may be obtained using standard cloning and screening techniques from a cDNA library derived from mRNA in cells of human bone marrow, (see for instance, Sambrook et al., Molecular Cloning: A Laboratory Manual, 2nd Ed., Cold Spring Harbor Laboratory Press, Cold Spring Harbor, N.Y. (1989)). Polynucleotides of the invention can also be obtained from natural sources such as genomic DNA libraries or can be synthesized using well known and commercially available techniques.

When polynucleotides of the present invention are used for the recombinant production of polypeptides of the present invention, the polynucleotide may include the coding sequence for the mature polypeptide, by itself, or the coding sequence for the mature polypeptide in reading frame with other coding sequences, such as those encoding a leader or secretory sequence, a pre-, or pro- or prepro-protein sequence, or other fusion peptide portions. For example, a marker sequence that facilitates purification of the fused polypeptide can be encoded. In certain preferred embodiments of this aspect of the invention, the marker sequence is a hexa-histidine peptide, as provided in the pQE vector (Qiagen, Inc.) and described in Gentz et al., Proc Natl Acad Sci USA (1989) 86:821-824, or is an HA tag. The polynucleotide may also contain non-coding 5′ and 3′ sequences, such as transcribed, non-translated sequences, splicing and polyadenylation signals, ribosome binding sites and sequences that stabilize mRNA.

Polynucleotides that are identical, or have sufficient identity to a polynucleotide sequence of SEQ ID NO:1, may be used as hybridization probes for cDNA and genomic DNA or as primers for a nucleic acid amplification reaction (for instance, PCR). Such probes and primers may be used to isolate full-length cDNAs and genomic clones encoding polypeptides of the present invention and to isolate cDNA and genomic clones of other genes (including genes encoding paralogs from human sources and orthologs and paralogs from species other than human) that have a high sequence similarity to SEQ ID NO:1, typically at least 95% identity. Preferred probes and primers will generally comprise at least 15 nucleotides, preferably, at least 30 nucleotides and may have at least 50, if not at least 100 nucleotides. Particularly preferred probes will have between 30 and 50 nucleotides. Particularly preferred primers will have between 20 and 25 nucleotides.

A polynucleotide encoding a polypeptide of the present invention, including homologs from species other than human, may be obtained by a process comprising the steps of screening a library under stringent hybridization conditions with a labeled probe having the sequence of SEQ ID NO: 1 or a fragment thereof, preferably of at least 15 nucleotides; and isolating full-length cDNA and genomic clones containing said polynucleotide sequence. Such hybridization techniques are well known to the skilled artisan. Preferred stringent hybridization conditions include overnight incubation at 42° C. in a solution comprising:50% formamide, 5×SSC (150 mM NaCl, 15 mM trisodium citrate), 50 mM sodium phosphate (pH 7.6), 5× Denhardt's solution, 10% dextran sulfate, and 20 microgram/ml denatured, sheared salmon sperm DNA; followed by washing the filters in 0.1×SSC at about 65° C. Thus the present invention also includes isolated polynucleotides, preferably with a nucleotide sequence of at least 100, obtained by screening a library under stringent hybridization conditions with a labeled probe having the sequence of SEQ ID NO:1 or a fragment thereof, preferably of at least 15 nucleotides.

The skilled artisan will appreciate that, in many cases, an isolated cDNA sequence will be incomplete, in that the region coding for the polypeptide does not extend all the way through to the 5′ terminus. This is a consequence of reverse transcriptase, an enzyme with inherently low “processivity” (a measure of the ability of the enzyme to remain attached to the template during the polymerization reaction), failing to complete a DNA copy of the mRNA template during first strand cDNA synthesis.

There are several methods available and well known to those skilled in the art to obtain full-length cDNAs, or extend short cDNAs, for example those based on the method of Rapid Amplification of cDNA ends (RACE) (see, for example, Frohman et al., Proc Nat Acad Sci USA 85, 8998-9002, 1988). Recent modifications of the technique, exemplified by the Marathon (trade mark) technology (Clontech Laboratories Inc.) for example, have significantly simplified the search for longer cDNAs. In the Marathon (trade mark) technology, cDNAs have been prepared from mRNA extracted from a chosen tissue and an ‘adaptor’ sequence ligated onto each end. Nucleic acid amplification (PCR) is then carried out to amplify the “missing” 5′ end of the cDNA using a combination of gene specific and adaptor specific oligonucleotide primers. The PCR reaction is then repeated using ‘nested’ primers, that is, primers designed to anneal within the amplified product (typically an adapter specific primer that anneals further 3′ in the adaptor sequence and a gene specific primer that anneals further 5′ in the known gene sequence). The products of this reaction can then be analyzed by DNA sequencing and a full-length cDNA constructed either by joining the product directly to the existing cDNA to give a complete sequence, or carrying out a separate full-length PCR using the new sequence information for the design of the 5′ primer.

Recombinant polypeptides of the present invention may be prepared by processes well known in the art from genetically engineered host cells comprising expression systems. Accordingly, in a further aspect, the present invention relates to expression systems comprising a polynucleotide or polynucleotides of the present invention, to host cells which are genetically engineered with such expression systems and to the production of polypeptides of the invention by recombinant techniques. Cell-free translation systems can also be employed to produce such proteins using RNAs derived from the DNA constructs of the present invention.

For recombinant production, host cells can be genetically engineered to incorporate expression systems or portions thereof for polynucleotides of the present invention. Polynucleotides may be introduced into host cells by methods described in many standard laboratory manuals, such as Davis et al., Basic Methods in Molecular Biology (1986) and Sambrook et al.(ibid). Preferred methods of introducing polynucleotides into host cells include, for instance, calcium phosphate transfection, DEAE-dextran mediated transfection, transvection, micro-injection, cationic lipid-mediated transfection, electroporation, transduction, scrape loading, ballistic introduction, or infection.

Representative examples of appropriate hosts include bacterial cells, such as Streptococci, Staphylococci, E. coli, Streptomyces and Bacillus subtilis cells; fungal cells, such as yeast cells and Aspergillus cells; insect cells such as Drosophila S2 and Spodoptera Sf9 cells; animal cells such as CHO, COS, HeLa, C127, 3T3, BHK, HEK 293 and Bowes melanoma cells; and plant cells.

A great variety of expression systems can be used, for instance, chromosomal, episomal and virus-derived systems, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. The expression systems may contain control regions that regulate as well as engender expression. Generally, any system or vector that is able to maintain, propagate, or express a polynucleotide to produce a polypeptide in a host may be used. The appropriate polynucleotide sequence may be inserted into an expression system by any of a variety of well-known and routine techniques, such as, for example, those set forth in Sambrook et al., (ibid). Appropriate secretion signals may be incorporated into the desired polypeptide to allow secretion of the translated protein into the lumen of the endoplasmic reticulum, the periplasmic space, or the extracellular environment. These signals may be endogenous to the polypeptide or they may be heterologous signals.

If a polypeptide of the present invention is to be expressed for use in screening assays, it is generally preferred that the polypeptide be produced at the surface of the cell. In this event, the cells may be harvested prior to use in the screening assay. If the polypeptide is secreted into the medium, the medium can be recovered in order to recover and purify the polypeptide. If produced intracellularly, the cells must first be lysed before the polypeptide is recovered.

Polypeptides of the present invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography is employed for purification. Well known techniques for refolding proteins may be employed to regenerate active conformation when the polypeptide is denatured during intracellular synthesis, isolation, and/or purification.

Polynucleotides of the present invention may be used as diagnostic reagents, through detecting mutations in the associated gene. Detection of a mutated form of the gene characterized by the polynucleotide of SEQ ID NO:1 in the cDNA or genomic sequence and which is associated with a dysfunction will provide a diagnostic tool that can add to, or define, a diagnosis of a disease of the invention, or susceptibility to a disease of the invention, which results from under-expression, over-expression or altered spatial or temporal expression of the gene. Individuals carrying mutations in the gene may be detected at the DNA level by a variety of techniques well known in the art.

Nucleic acids for diagnosis may be obtained from a subject's cells, such as from blood, urine, saliva, tissue biopsy or autopsy material. The genomic DNA may be used directly for detection or it may be amplified enzymatically by using PCR, preferably RT-PCR, or other amplification techniques prior to analysis. RNA or cDNA may also be used in similar fashion. Deletions and insertions can be detected by a change in size of the amplified product in comparison to the normal genotype. Point mutations can be identified by hybridizing amplified DNA to labeled AXOR35 nucleotide sequences. Perfectly matched sequences can be distinguished from mismatched duplexes by RNase digestion or by differences in melting temperatures. DNA sequence difference may also be detected by alterations in the electrophoretic mobility of DNA fragments in gels, with or without denaturing agents, or by direct DNA sequencing (see, for instance, Myers et al., Science (1985) 230:1242). Sequence changes at specific locations may also be revealed by nuclease protection assays, such as RNase and S1 protection or the chemical cleavage method (see Cotton et al., Proc Natl Acad Sci USA (1985) 85: 4397-4401).

An array of oligonucleotides probes comprising AXOR35 polynucleotide sequence or fragments thereof can be constructed to conduct efficient screening of e.g., genetic mutations. Such arrays are preferably high density arrays or grids. Array technology methods are well known and have general applicability and can be used to address a variety of questions in molecular genetics including gene expression, genetic linkage, and genetic variability, see, for example, M. Chee et al., Science, 274, 610-613 (1996) and other references cited therein.

Detection of abnormally decreased or increased levels of polypeptide or mRNA expression may also be used for diagnosing or determining susceptibility of a subject to a disease of the invention. Decreased or increased expression can be measured at the RNA level using any of the methods well known in the art for the quantitation of polynucleotides, such as, for example, nucleic acid amplification, for instance PCR, RT-PCR, RNase protection, Northern blotting and other hybridization methods. Assay techniques that can be used to determine levels of a protein, such as a polypeptide of the present invention, in a sample derived from a host are well-known to those of skill in the art. Such assay methods include radio-immunoassays, competitive-binding assays, Western Blot analysis and ELISA assays.

Thus in another aspect, the present invention relates to a diagnostic kit comprising:

(a) a polynucleotide of the present invention, preferably the nucleotide sequence of SEQ ID NO:1, or a fragment or an RNA transcript thereof;

(b) a nucleotide sequence complementary to that of (a);

(c) a polypeptide of the present invention, preferably the polypeptide of SEQ ID NO:2 or a fragment thereof; or

(d) an antibody to a polypeptide of the present invention, preferably to the polypeptide of SEQ ID NO:2.

It will be appreciated that in any such kit, (a), (b), (c) or (d) may comprise a substantial component. Such a kit will be of use in diagnosing a disease or susceptibility to a disease, particularly diseases of the invention, amongst others.

The polynucleotide sequences of the present invention are valuable for chromosome localization studies. The sequence is specifically targeted to, and can hybridize with, a particular location on an individual human chromosome. The mapping of relevant sequences to chromosomes according to the present invention is an important first step in correlating those sequences with gene associated disease. Once a sequence has been mapped to a precise chromosomal location, the physical position of the sequence on the chromosome can be correlated with genetic map data. Such data are found in, for example, V. McKusick, Mendelian Inheritance in Man (available on-line through Johns Hopkins University Welch Medical Library). The relationship between genes and diseases that have been mapped to the same chromosomal region are then identified through linkage analysis (co-inheritance of physically adjacent genes). Precise human chromosomal localizations for a genomic sequence (gene fragment etc.) can be determined using Radiation Hybrid (RH) Mapping (Walter, M. Spillett, D., Thomas, P., Weissenbach, J., and Goodfellow, P., (1994). A method for constructing radiation hybrid maps of whole genomes, Nature Genetics 7, 22-28). A number of RH panels are available from Research Genetics (Huntsville, Ala., USA) e.g. the GeneBridge4 RH panel (Hum Mol Genet 1996 Mar; 5(3):339-46. A radiation hybrid map of the human genome. Gyapay G, Schmitt K, Fizames C, Jones H, Vega-Czarny N, Spillett D, Muselet D, Prud'Homme J F, Dib C, Auffray C, Morissette J, Weissenbach J, Goodfellow PN). To determine the chromosomal location of a gene using this panel, 93 PCRs are performed using primers designed from the gene of interest on RH DNAs. Each of these DNAs contains random human genomic fragments maintained in a hamster background (human/hamster hybrid cell lines). These PCRs result in 93 scores indicating the presence or absence of the PCR product of the gene of interest. These scores are compared with scores created using PCR products from genomic sequences of known location. This comparison is conducted at http://www.genome.wi.mit.edu.

The polynucleotide sequences of the present invention are also valuable tools for tissue expression studies. Such studies allow the determination of expression patterns of polynucleotides of the present invention which may give an indication as to the expression patterns of the encoded polypeptides in tissues, by detecting the mRNAs that encode them. The techniques used are well known in the art and include in situ hybridization techniques to clones arrayed on a grid, such as cDNA microarray hybridization (Schena et al, Science, 270, 467-470, 1995 and Shalon et al, Genome Res, 6, 639-645, 1996) and nucleotide amplification techniques such as PCR. A preferred method uses the TAQMAN (Trade mark) technology available from Perkin Elmer. Results from these studies can provide an indication of the normal function of the polypeptide in the organism. In addition, comparative studies of the normal expression pattern of mRNAs with that of mRNAs encoded by an alternative form of the same gene (for example, one having an alteration in polypeptide coding potential or a regulatory mutation) can provide valuable insights into the role of the polypeptides of the present invention, or that of inappropriate expression thereof in disease. Such inappropriate expression may be of a temporal, spatial or simply quantitative nature.

By Taqman, the polypeptides of the present invention are expressed in peripheral blood monocytes and bone marrow. By in situ hybridization, the expression of AXOR35 was detected in lymphocytes, macrophages, eosinophils, and neutrophils in asthamtic lung, but not in normal lung. Thus, the present invention relates a method of inhibiting or promoting the function of lymphocytes, macrophages, eosinophils, or neutrophils in diseased tissue, such as, but not limited to, asthmatic lung, by administering to a patient in need thereof AXOR35 agonists or antagonists. In further aspect, the present invention relates a method of inhibiting or promoting the function of lymphocytes, macrophages, eosinophils, or neutrophils in diseased tissue, such as, but not limited to, asthmatic lung, by administering to a patient in need thereof AXOR35 agonists or antagonists identified by any of the screening methods described herein. The present invention further relates to a method of agonizing or antagonizing AXOR35 in order to inhibit or promote the function of lymphocytes, macrophages, eosinophils, or neutrophils in diseased tissue, such as, but not limited to, asthmatic lung, by administering to a patient in need thereof AXOR35 agonists or antagonists. Moreover, the present invention relates to a method of agonizing or antagonizing AXOR35 in order to inhibit or promote the function of lymphocytes, macrophages, eosinophils, or neutrophils in diseased tissue, such as, but not limited to, asthmatic lung, by administering to a patient in need thereof AXOR35 agonists or antagonists identified by any of the screening methods described herein.

A further aspect of the present invention relates to antibodies. The polypeptides of the invention or their fragments, or cells expressing them, can be used as immunogens to produce antibodies that are immunospecific for polypeptides of the present invention. The term “immunospecific” means that the antibodies have substantially greater affinity for the polypeptides of the invention than their affinity for other related polypeptides in the prior art.

Antibodies generated against polypeptides of the present invention may be obtained by administering the polypeptides or epitope-bearing fragments, or cells to an animal, preferably a non-human animal, using routine protocols. For preparation of monoclonal antibodies, any technique which provides antibodies produced by continuous cell line cultures can be used. Examples include the hybridoma technique (Kohler, G. and Milstein, C., Nature (1975) 256:495-497), the trioma technique, the human B-cell hybridoma technique (Kozbor et al., Immunology Today (1983) 4:72) and the EBV-hybridoma technique (Cole et al., Monoclonal Antibodies and Cancer Therapy, 77-96, Alan R. Liss, Inc., 1985).

Techniques for the production of single chain antibodies, such as those described in U.S. Pat. No.4,946,778, can also be adapted to produce single chain antibodies to polypeptides of this invention. Also, transgenic mice, or other organisms, including other mammals, may be used to express humanized antibodies.

The above-described antibodies may be employed to isolate or to identify clones expressing the polypeptide or to purify the polypeptides by affinity chromatography. Antibodies against polypeptides of the present invention may also be employed to treat diseases of the invention, amongst others.

Polypeptides and polynucleotides of the present invention may also be used as vaccines. Accordingly, in a further aspect, the present invention relates to a method for inducing an immunological response in a mammal that comprises inoculating the mammal with a polypeptide of the present invention, adequate to produce antibody and/or T cell immune response, including, for example, cytokine-producing T cells or cytotoxic T cells, to protect said animal from disease, whether that disease is already established within the individual or not. An immunological response in a mammal may also be induced by a method comprises delivering a polypeptide of the present invention via a vector directing expression of the polynucleotide and coding for the polypeptide in vivo in order to induce such an immunological response to produce antibody to protect said animal from diseases of the invention. One way of administering the vector is by accelerating it into the desired cells as a coating on particles or otherwise. Such nucleic acid vector may comprise DNA, RNA, a modified nucleic acid, or a DNA/RNA hybrid. For use a vaccine, a polypeptide or a nucleic acid vector will be normally provided as a vaccine formulation (composition). The formulation may further comprise a suitable carrier. Since a polypeptide may be broken down in the stomach, it is preferably administered parenterally (for instance, subcutaneous, intra-muscular, intravenous, or intra-dermal injection). Formulations suitable for parenteral administration include aqueous and non-aqueous sterile injection solutions that may contain anti-oxidants, buffers, bacteriostats and solutes that render the formulation instonic with the blood of the recipient; and aqueous and non-aqueous sterile suspensions that may include suspending agents or thickening agents. The formulations may be presented in unit-dose or multi-dose containers, for example, sealed ampoules and vials and may be stored in a freeze-dried condition requiring only the addition of the sterile liquid carrier immediately prior to use. The vaccine formulation may also include adjuvant systems for enhancing the immunogenicity of the formulation, such as oil-in water systems and other systems known in the art. The dosage will depend on the specific activity of the vaccine and can be readily determined by routine experimentation.

Polypeptides of the present invention have one or more biological functions that are of relevance in one or more disease states, in particular the diseases of the invention hereinbefore mentioned. It is therefore useful to identify compounds that stimulate or inhibit the function or level of the polypeptide. Accordingly, in a further aspect, the present invention provides for a method of screening compounds to identify those that stimulate or inhibit the function or level of the polypeptide. Such methods identify agonists or antagonists that may be employed for therapeutic and prophylactic purposes for such diseases of the invention as hereinbefore mentioned. Compounds may be identified from a variety of sources, for example, cells, cell-free preparations, chemical libraries, collections of chemical compounds, and natural product mixtures. Such agonists or antagonists so-identified may be natural or modified substrates, ligands, receptors, enzymes, etc., as the case may be, of the polypeptide; a structural or functional mimetic thereof (see Coligan et al., Current Protocols in Immunology 1(2):Chapter 5 (1991)) or a small molecule. Such small molecules preferably have a molecular weight below 2,000 daltons, more preferably between 300 and 1,000 daltons, and most preferably between 400 and 700 daltons. It is preferred that these small molecules are organic molecules.

The screening method may simply measure the binding of a candidate compound to the polypeptide, or to cells or membranes bearing the polypeptide, or a fusion protein thereof, by means of a label directly or indirectly associated with the candidate compound. Alternatively, the screening method may involve measuring or detecting (qualitatively or quantitatively) the competitive binding of a candidate compound to the polypeptide against a labeled competitor (e.g. agonist or antagonist). Further, these screening methods may test whether the candidate compound results in a signal generated by activation or inhibition of the polypeptide, using detection systems appropriate to the cells bearing the polypeptide. Inhibitors of activation are generally assayed in the presence of a known agonist and the effect on activation by the agonist by the presence of the candidate compound is observed. Further, the screening methods may simply comprise the steps of mixing a candidate compound with a solution containing a polypeptide of the present invention, to form a mixture, measuring a AXOR35 activity in the mixture, and comparing the AXOR35 activity of the mixture to a control mixture which contains no candidate compound.

Polypeptides of the present invention may be employed in conventional low capacity screening methods and also in high-throughput screening (HTS) formats. Such HTS formats include not only the well-established use of 96- and, more recently, 384-well micotiter plates but also emerging methods such as the nanowell method described by Schullek et al, Anal Biochem., 246, 20-29, (1997).

Fusion proteins, such as those made from Fc portion and AXOR35 polypeptide, as hereinbefore described, can also be used for high-throughput screening assays to identify antagonists for the polypeptide of the present invention (see D. Bennett et al., J Mol Recognition, 8:52-58 (1995); and K. Johanson et al., J Biol Chem, 270(16):9459-9471 (1995)).

We have now discovered that histamine or a histamine-like compound is a ligand for AXOR35 polypeptide. The AXOR35 polypeptide of the present invention may be employed in a process for screening for compounds which bind to and activate the AXOR35 polypeptides of the present invention (called agonists), or inhibit the interaction of the AXOR35 polypeptides with receptor ligands (called antagonists).

In general, such screening procedures involve providing appropriate cells which express the receptor polypeptide of the present invention on the surface thereof. Such cells include cells from mammals, yeast, Drosophila or E. coli. In particular, a polynucleotide encoding the receptor of the present invention is employed to transfect cells to thereby express the AXOR35 polypeptide. The expressed receptor is then contacted with a test compound to observe binding, stimulation or inhibition of a functional response.

One such screening procedure involves the use of melanophores which are transfected to express the AXOR35 polypeptide of the present invention. Such a screening technique is described in PCT WO 92/01810, published Feb. 6, 1992. Such an assay may be employed to screen for a compound which inhibits activation of the receptor polypeptide of the present invention by contacting the melanophore cells which encode the receptor with both the receptor ligand, such as histamine or a histamine-like compound, and a compound to be screened. Inhibition of the signal generated by the ligand indicates that a compound is a potential antagonist for the receptor, i.e., inhibits activation of the receptor.

The technique may also be employed for screening of compounds which activate the receptor by contacting such cells with compounds to be screened and determining whether such compound generates a signal, i.e., activates the receptor. Other screening techniques include the use of cells which express the AXOR35 polypeptide (for example, transfected CHO cells) in a system which measures extracellular pH changes caused by receptor activation. In this technique, compounds may be contacted with cells expressing the receptor polypeptide of the present invention. A second messenger response, e.g., signal transduction or pH changes, is then measured to determine whether the potential compound activates or inhibits the receptor.

Another screening technique involves expressing the AXOR35 polypeptide in which the receptor is linked to phospholipase C or D. Representative examples of such cells include, but are not limited to, endothelial cells, smooth muscle cells, and embryonic kidney cells. The screening may be accomplished as hereinabove described by detecting activation of the receptor or inhibition of activation of the receptor from the phospholipase second signal.

Another method involves screening for compounds which are antagonists or agonists by determining inhibition of binding of labeled ligand, such as histamine or a histamine-like compound, to cells which have the receptor on the surface thereof, or cell membranes containing the receptor. Such a method involves transfecting a cell (such as eukaryotic cell) with DNA encoding the AXOR35 polypeptide such that the cell expresses the receptor on its surface. The cell is then contacted with a potential antagonist or agonist in the presence of a labeled form of a ligand, such as histamine or a histamine-like compound. The ligand can be labeled, e.g., by radioactivity. The amount of labeled ligand bound to the receptors is measured, e.g., by measuring radioactivity associated with transfected cells or membrane from these cells. If the compound binds to the receptor, the binding of labeled ligand to the receptor is inhibited as determined by a reduction of labeled ligand which binds to the receptors. This method is called binding assay.

Another screening procedure involves the use of mammalian or amphibian cells (CHO, HEK 293, Xenopus Oocytes, RBL-2H3, etc) which are transfected to express the receptor of interest. The cells are loaded with an indicator dye that produces a fluorescent signal when bound to calcium, and the cells are contacted with a test substance and a receptor agonist, such as histamine or a histamine-like compound. Any change in fluorescent signal is measured over a defined period of time using, for example, a fluorescence spectrophotometer or a fluorescence imaging plate reader. A change in the fluorescence signal pattern generated by the ligand indicates that a compound is a potential antagonist or agonist for the receptor.

Another screening procedure involves use of mammalian or amphibian cells (CHO, HEK293, Xenopus Oocytes, RBL-2H3, etc.) which are transfected to express the receptor of interest, and which are also transfected with a reporter gene construct that is coupled to activation of the receptor (for example, luciferase, beta-galactosidase, or beta-lactamase behind an appropriate promoter). The cells are contacted with a test substance and the receptor agonist (ligand), such as histamine or a histamine-like compound, and the signal produced by the reporter gene is measured after a defined period of time. A suitable substrate for the reporter gene product is added prior to measurement of the signal. For example, luciferin could be added to measure luciferase activity, or CCF2/AM could be added to measure beta-lactamase activity (Zlokarnik et al, Science 1998, 279, 84-88). The signal can be measured using a luminometer, spectrophotometer, fluorimeter, or other such instrument appropriate for the specific reporter construct used. Change of the signal generated by the ligand indicates that a compound is a potential antagonist or agonist for the receptor.

Another screening technique for antagonists or agonists involves introducing polynucleotide (RNA or DNA) encoding the AXOR35 polypeptide into Xenopus oocytes (or CHO, HEK 293, RBL-2H3. etc.) to express the receptor. The receptor oocytes are then contacted with the receptor ligand, such as histamine or a histamine-like compound, and a compound to be screened. Inhibition or activation of the receptor is then determined by detecting a change in a signal, such as, cAMP, calcium, proton, or other ions.

For example, one method involves screening for AXOR35 polypeptide inhibitors by determining inhibition or stimulation of AXOR35 polypeptide-mediated cAMP and/or adenylate cyclase accumulation or dimunition. Such a method involves transiently or stably transfecting a eukaryotic cell with AXOR35 polypeptide receptor to express the receptor on the cell surface. The cell is then exposed to potential antagonists or agonists in the presence of AXOR35 polypeptide ligand, such as histamine or a histamine-like compound. The changes in levels of cAMP is then measured over a defined period of time, for example, by radio-immuno or protein binding assays (for example using Flashplates or a scintillation proximity assay). Changes in cAMP levels can also be determined by directly measuring the activity of the enzyme, adenylyl cyclase, in broken cell preparations. If the potential antagonist or agonist binds the receptor, and thus inhibits AXOR35 polypeptide-ligand binding, the levels of AXOR35 polypeptide-mediated cAMP, or adenylate cyclase activity, will be reduced or increased.

Another screening method for agonists and antagonists relies on the endogenous pheromone response pathway in the yeast, Saccharomyces cerevisiae. Heterothallic strains of yeast can exist in two mitotically stable haploid mating types, MATa and MATa. Each cell type secretes a small peptide hormone that binds to a G-protein coupled receptor on opposite mating-type cells which triggers a MAP kinase cascade leading to G1 arrest as a prelude to cell fusion. Genetic alteration of certain genes in the pheromone response pathway can alter the normal response to pheromone, and heterologous expression and coupling of human G-protein coupled receptors and humanized G-protein subunits in yeast cells devoid of endogenous pheromone receptors can be linked to downstream signaling pathways and reporter genes (e.g., U.S. Pat. Nos. 5,063,154; 5,482,835; 5,691,188). Such genetic alterations include, but are not limited to, (i) deletion of the STE2 or STE3 gene encoding the endogenous G-protein coupled pheromone receptors; (ii) deletion of the FAR1 gene encoding a protein that normally associates with cyclin-dependent kinases leading to cell cycle arrest; and (iii) construction of reporter genes fused to the FUS1 gene promoter (where FUS1 encodes a membrane-anchored glycoprotein required for cell fusion). Downstream reporter genes can permit either a positive growth selection (e.g., histidine prototrophy using the FUS1-HIS3 reporter), or a colorimetric, fluorimetric or spectrophotometric readout, depending on the specific reporter construct used (e.g., b-galactosidase induction using a FUS1-LacZ reporter).

The yeast cells can be further engineered to express and secrete small peptides from random peptide libraries, some of which can permit autocrine activation of heterologously expressed human (or mammalian) G-protein coupled receptors (Broach, J. R. and Thorner, J. Nature 384: 14-16, 1996; Manfredi et al., Mol. Cell. Biol. 16: 4700-4709, 1996). This provides a rapid direct growth selection (e.g, using the FUS1-HIS3 reporter) for surrogate peptide agonists that activate characterized or orphan receptors. Alternatively, yeast cells that functionally express human (or mammalian) G-protein coupled receptors linked to a reporter gene readout (e.g., FUS1-LacZ) can be used as a platform for high-throughput screening of known ligands, fractions of biological extracts and libraries of chemical compounds for either natural or surrogate ligands. Functional agonists of sufficient potency (whether natural or surrogate) can be used as screening tools in yeast cell-based assays for identifying G-protein coupled receptor antagonists. For example, agonists will promote growth of a cell with FUS-HIS3 reporter or give positive readout for a cell with FUS1-LacZ. However, a candidate compound which inhibits growth or negates the positive readout induced by an agonist is an antagonist. For this purpose, the yeast system offers advantages over mammalian expression systems due to its ease of utility and null receptor background (lack of endogenous G-protein coupled receptors) which often interferes with the ability to identify agonists or antagonists.

The present invention also provides a method for identifying new ligands not known to be capable of binding to an AXOR35 polypeptides. The screening assays described above for identifying agonists may be used to identify new ligands.

The present invention also contemplates agonists and antagonists obtainable from any of the above described screening methods.

Examples of potential AXOR35 polypeptide receptor antagonists include peptidomimetics, synthetic organic molecules, natural products, antibodies, etc. which bind to the receptor but do not elicit a second messenger response such that the activity of the receptor is prevented.

Potential antagonists also include proteins which are closely related to the ligand of the AXOR35 polypeptide receptor, i.e. a fragment of the ligand, which have lost biological function, and when they bind to the AXOR35 polypeptide receptor, elicit no response.

In one preferred embodiment, the present invention relates to particular antagonists and further agonists (apart from histamine and histamine-like compounds as defined below) identified by the screening methods described herein as shown in Table I and Table II.

TABLE I

| | Compounds are prepared as described in |
|---|---|
| | the references cited below, or provided in |
| Agonists^{a} | the Examples |
| 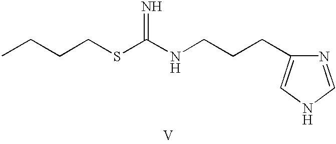 | US3736331, US3881015 |
| 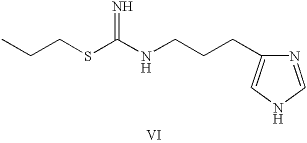 | US3736331, US3881015 |
| 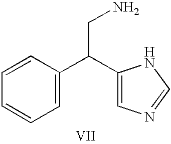 | See Example 10 |
| 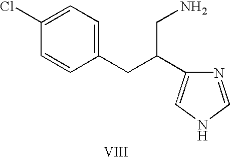 | See Example 11 |
| 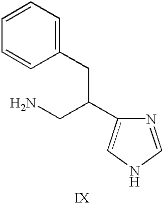 | J. Med. Chem 1982 25(10), p.1168. GB 1341375 |

^{a}The compounds above have EC50 range of 0.0105-0.452 uM. The assay for EC50 is provided in Example 9.

TABLE II

| | Compounds are prepared as described in |
|---|---|
| | the references cited below, or provided in |
| Antagonist^{b} | the Examples |
| 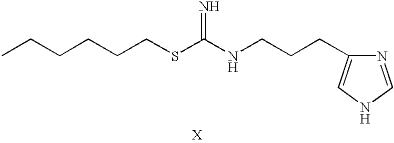 | US3736331, US3881015 |
| 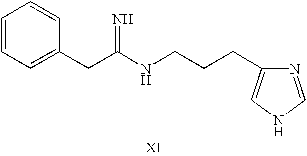 | US3734924, US3881016 |
| 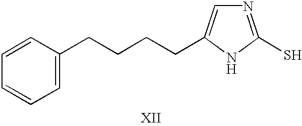 | US4798843 |
| 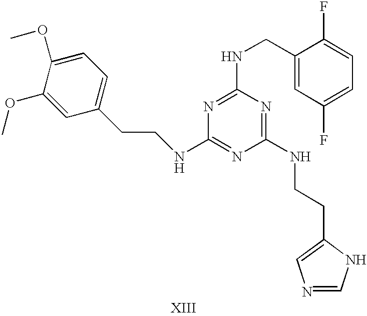 | See Example 12 |

^{b}The compounds above have IC50 range of 0.195-0.323 uM. The assay for IC50 is provided in Example 9.

A potential antagonist also includes an antisense construct prepared through the use of antisense technology. Antisense technology can be used to control gene expression through triple-helix formation or antisense DNA or RNA, both methods of which are based on binding of a polynucleotide to DNA or RNA. For example, the 5′ coding portion of the polynucleotide sequence, which encodes for the mature polypeptides of the present invention, is used to design an antisense RNA oligonucleotide of from about 10 to 40 base pairs in length. A DNA oligonucleotide is designed to be complementary to a region of the gene involved in transcription (triple helix -see Lee, et al. Nucl. Acids Res., 6: 3073 (1979); Cooney, et al, Science, 241: 456 (1988); and Dervan, et al., Science, 251: 1360 (1991)), thereby preventing transcription and production of a AXOR35 polypeptide. The antisense RNA oligonucleotide hybridizes to the mRNA in vivo and blocks translation of the mRNA molecule to a AXOR35 polypeptide (antisense—Okano, J., Neurochem., 56: 560 (1991); OLIGODEOXYNUCLEOTIDES AS ANTISENSE INHIBITORS OF GENE EXPRESSION, CRC Press, Boca Raton, Fla. (1988)). The oligonucleotides described above can also be delivered to cells such that the antisense RNA or DNA may be expressed in vivo to inhibit production of an AXOR35 polypeptide.

Thus in another aspect, the present invention relates to a screening kit for identifying agonists, antagonists, and ligands for AXOR35 polypeptides, which comprises:

(a) an AXOR35 polypeptide, preferably that of SEQ ID NO:2; and further preferably comprises labeled or unlabeled histamine or a histamine-like compound;

(b) a recombinant cell expressing a AXOR35 polypeptide, preferably that of SEQ ID NO:2; and further preferably comprises labeled or unlabeled histamine or a histamine-like compound; or

(c) a cell membrane expressing AXOR35 polypeptide; preferably that of SEQ ID NO:2; and further preferably comprises labeled or unlabeled histamine or a histamine-like compound.

It will be appreciated that in any such kit, (a), (b), or (c) may comprise a substantial component.

The polynucleotides, polypeptides and antibodies to the polypeptide of the present invention may also be used to configure screening methods for detecting the effect of added compounds on the production of mRNA and polypeptide in cells. For example, an ELISA assay may be constructed for measuring secreted or cell associated levels of polypeptide using monoclonal and polyclonal antibodies by standard methods known in the art. This can be used to discover agents which may inhibit or enhance the production of polypeptide (also called antagonist or agonist, respectively) from suitably manipulated cells or tissues.

The polypeptide may be used to identify membrane bound or soluble receptors, if any, through standard receptor binding techniques known in the art. These include, but are not limited to, ligand binding and crosslinking assays in which the polypeptide is labeled with a radioactive isotope (for instance, ¹²⁵I), chemically modified (for instance, biotinylated), or fused to a peptide sequence suitable for detection or purification, and incubated with a source of the putative receptor (cells, cell membranes, cell supernatants, tissue extracts, bodily fluids). Other methods include biophysical techniques such as surface plasmon resonance and spectroscopy. These screening methods may also be used to identify agonists and antagonists of the polypeptide which compete with the binding of the polypeptide to its receptors, if any. Standard methods for conducting such assays are well understood in the art.

Examples of potential polypeptide antagonists include antibodies or, in some cases, oligonucleotides or proteins which are closely related to the ligands, substrates, receptors, enzymes, etc., as the case may be, of the polypeptide, e.g., a fragment of the ligands, substrates, receptors, enzymes, etc.; or small molecules which bind to the polypeptide of the present invention but do not elicit a response, so that the activity of the polypeptide is prevented.

Thus, in another aspect, the present invention relates to a screening kit for identifying agonists, antagonists, ligands, receptors, substrates, enzymes, etc. for polypeptides of the present invention; or compounds which decrease or enhance the production of such polypeptides, which comprises:

(a) a polypeptide of the present invention;

(b) a recombinant cell expressing a polypeptide of the present invention;

(c) a cell membrane expressing a polypeptide of the present invention; or

(d) antibody to a polypeptide of the present invention; which polypeptide is preferably that of SEQ ID NO:2.

It will be appreciated that in any such kit, (a), (b), (c) or (d) may comprise a substantial component.

It will be readily appreciated by the skilled artisan that a polypeptide of the present invention may also be used in a method for the structure-based design of an agonist, antagonist or inhibitor of the polypeptide, by:

(a) determining in the first instance the three-dimensional structure of the polypeptide;

(b) deducing the three-dimensional structure for the likely reactive or binding site(s) of an agonist, antagonist or inhibitor;

(c) synthesizing candidate compounds that are predicted to bind to or react with the deduced binding or reactive site; and

(d) testing whether the candidate compounds are indeed agonists, antagonists or inhibitors. It will be further appreciated that this will normally be an interactive process.

If the activity of the polypeptide is in excess, several approaches are available. One approach comprises administering to a subject in need thereof an inhibitor compound (antagonist) as hereinabove described, optionally in combination with a pharmaceutically acceptable carrier, in an amount effective to inhibit the function of the polypeptide, such as, for example, by blocking the binding of ligands, substrates, receptors, enzymes, etc., or by inhibiting a second signal, and thereby alleviating the abnormal condition. In another approach, soluble forms of the polypeptides still capable of binding the ligand, substrate, enzymes, receptors, etc. in competition with endogenous polypeptide may be administered. Typical examples of such competitors include fragments of the AXOR35 polypeptide.

In still another approach, expression of the gene encoding endogenous AXOR35 polypeptide can be inhibited using expression blocking techniques. Known such techniques involve the use of antisense sequences, either internally generated or separately administered (see, for example, O'Connor, J Neurochem (1991) 56:560 in Oligodeoxynucleotides as Antisense Inhibitors of Gene Expression, CRC Press, Boca Raton, Fla. (1988)). Alternatively, oligonucleotides which form triple helices with the gene can be supplied (see, for example, Lee et al., Nucleic Acids Res (1979) 6:3073; Cooney et al., Science (1988) 241:456; Dervan et al., Science (1991) 251:1360). These oligomers can be administered per se or the relevant oligomers can be expressed in vivo.

For treating abnormal conditions related to an under-expression of AXOR35 and its activity, several approaches are also available. One approach comprises administering to a subject a therapeutically effective amount of a compound which activates a polypeptide of the present invention, i.e., an agonist as described above, in combination with a pharmaceutically acceptable carrier, to thereby alleviate the abnormal condition. Alternatively, gene therapy may be employed to effect the endogenous production of AXOR35 by the relevant cells in the subject. For example, a polynucleotide of the invention may be engineered for expression in a replication defective retroviral vector, as discussed above. The retroviral expression construct may then be isolated and introduced into a packaging cell transduced with a retroviral plasmid vector containing RNA encoding a polypeptide of the present invention such that the packaging cell now produces infectious viral particles containing the gene of interest. These producer cells may be administered to a subject for engineering cells in vivo and expression of the polypeptide in vivo. For an overview of gene therapy, see Chapter 20, Gene Therapy and other Molecular Genetic-based Therapeutic Approaches, (and references cited therein) in Human Molecular Genetics, T Strachan and A P Read, BIOS Scientific Publishers Ltd (1996). Another approach is to administer a therapeutic amount of a polypeptide of the present invention in combination with a suitable pharmaceutical carrier.

In a further aspect, the present invention provides for pharmaceutical compositions comprising a therapeutically effective amount of a polypeptide, such as the soluble form of a polypeptide of the present invention, agonist/antagonist peptide or small molecule compound, in combination with a pharmaceutically acceptable carrier or excipient. Such carriers include, but are not limited to, saline, buffered saline, dextrose, water, glycerol, ethanol, and combinations thereof. The invention further relates to pharmaceutical packs and kits comprising one or more containers filled with one or more of the ingredients of the aforementioned compositions of the invention. Polypeptides and other compounds of the present invention may be employed alone or in conjunction with other compounds, such as therapeutic compounds.

The composition will be adapted to the route of administration, for instance by a systemic or an oral route. Preferred forms of systemic administration include injection, typically by intravenous injection. Other injection routes, such as subcutaneous, intramuscular, or intraperitoneal, can be used. Alternative means for systemic administration include transmucosal and transdermal administration using penetrants such as bile salts or fusidic acids or other detergents. In addition, if a polypeptide or other compounds of the present invention can be formulated in an enteric or an encapsulated formulation, oral administration may also be possible. Administration of these compounds may also be topical and/or localized, in the form of salves, pastes, gels, and the like.

The dosage range required depends on the choice of peptide or other compounds of the present invention, the route of administration, the nature of the formulation, the nature of the subject's condition, and the judgment of the attending practitioner. Suitable dosages, however, are in the range of 0.1-100 μg/kg of subject. Wide variations in the needed dosage, however, are to be expected in view of the variety of compounds available and the differing efficiencies of various routes of administration. For example, oral administration would be expected to require higher dosages than administration by intravenous injection. Variations in these dosage levels can be adjusted using standard empirical routines for optimization, as is well understood in the art.

Polypeptides used in treatment can also be generated endogenously in the subject, in treatment modalities often referred to as “gene therapy” as described above. Thus, for example, cells from a subject may be engineered with a polynucleotide, such as a DNA or RNA, to encode a polypeptide ex vivo, and for example, by the use of a retroviral plasmid vector. The cells are then introduced into the subject.

Polynucleotide and polypeptide sequences form a valuable information resource with which to identify further sequences of similar homology. This is most easily facilitated by storing the sequence in a computer readable medium and then using the stored data to search a sequence database using well known searching tools, such as GCC. Accordingly, in a further aspect, the present invention provides for a computer readable medium having stored thereon a polynucleotide comprising the sequence of SEQ ID NO:1 and/or a polypeptide sequence encoded thereby.

Screening methods may also involve the use of transgenic technology and AXOR35 gene. The art of constructing transgenic animals is well established. For example, the AXOR35 gene may be introduced through microinjection into the male pronucleus of fertilized oocytes, retroviral transfer into pre- or post-implantation embryos, or injection of genetically modified, such as by electroporation, embryonic stem cells into host blastocysts. Particularly useful transgenic animals are so-called “knock-in” animals in which an animal gene is replaced by the human equivalent within the genome of that animal. Knock-in transgenic animals are useful in the drug discovery process, for target validation, where the compound is specific for the human target. Other useful transgenic animals are so-called “knock-out” animals in which the expression of the animal ortholog of a polypeptide of the present invention and encoded by an endogenous DNA sequence in a cell is partially or completely annulled. The gene knock-out may be targeted to specific cells or tissues, may occur only in certain cells or tissues as a consequence of the limitations of the technology, or may occur in all, or substantially all, cells in the animal. Transgenic animal technology also offers a whole animal expression-cloning system in which introduced genes are expressed to give large amounts of polypeptides of the present invention.

Glossary

The following definitions are provided to facilitate understanding of certain terms used frequently hereinbefore.

“Antibodies” as used herein includes polyclonal and monoclonal antibodies, chimeric, single chain, and humanized antibodies, as well as Fab fragments, including the products of a Fab or other immunoglobulin expression library.

“Isolated” means altered “by the hand of man” from its natural state, i.e., if it occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated,” as the term is employed herein. Moreover, a polynucleotide or polypeptide that is introduced into an organism by transformation, genetic manipulation or by any other recombinant method is “isolated” even if it is still present in said organism, which organism may be living or non-living.

“Polynucleotide” generally refers to any polyribonucleotide (RNA) or polydeoxribonucleotide (DNA), which may be unmodified or modified RNA or DNA. “Polynucleotides” include, without limitation, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term “polynucleotide” also includes DNAs or RNAs containing one or more modified bases and DNAs or RNAs with backbones modified for stability or for other reasons. “Modified” bases include, for example, tritylated bases and unusual bases such as inosine. A variety of modifications may be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

“Polypeptide” refers to any polypeptide comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids. “Polypeptides” include amino acid sequences modified either by natural processes, such as post-translational processing, or by chemical modification techniques that are well known in the art. Such modifications are well described in basic texts and in more detailed monographs, as well as in a voluminous research literature. Modifications may occur anywhere in a polypeptide, including the peptide backbone, the amino acid side-chains and the amino or carboxyl termini. It will be appreciated that the same type of modification may be present to the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from post-translation natural processes or may be made by synthetic methods. Modifications include acetylation, acylation, ADP-ribosylation, amidation, biotinylation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination (see, for instance, Proteins-Structure and Molecular Properties, 2nd Ed., T. E. Creighton, W. H. Freeman and Company, New York, 1993; Wold, F., Post-translational Protein Modifications: Perspectives and Prospects, 1-12, in Post-translational Covalent Modification of Proteins, B. C. Johnson, Ed., Academic Press, New York, 1983; Seifter et al., “Analysis for protein modifications and nonprotein cofactors”, Meth Enzymol, 182, 626-646, 1990, and Rattan et al., “Protein Synthesis: Post-tranlational Modifications and Aging”, Ann NY Acad Sci, 663, 48-62, 1992).

“Fragment” of a polypeptide sequence refers to a polypeptide sequence that is shorter than the reference sequence but that retains essentially the same biological function or activity as the reference polypeptide. “Fragment” of a polynucleotide sequence refers to a polynucleotide sequence that is shorter than the reference sequence of SEQ ID NO:1.

“Variant” refers to a polynucleotide or polypeptide that differs from a reference polynucleotide or polypeptide, but retains the essential properties thereof. A typical variant of a polynucleotide differs in nucleotide sequence from the reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from the reference polypeptide. Generally, alterations are limited so that the sequences of the reference polypeptide and the variant are closely similar overall and, in many regions, identical. A variant and reference polypeptide may differ in amino acid sequence by one or more substitutions, insertions, deletions in any combination. A substituted or inserted amino acid residue may or may not be one encoded by the genetic code. Typical conservative substitutions include Gly, Ala; Val, Ile, Leu; Asp, Glu; Asn, Gln; Ser, Thr; Lys, Arg; and Phe and Tyr. A variant of a polynucleotide or polypeptide may be naturally occurring such as an allele, or it may be a variant that is not known to occur naturally. Non-naturally occurring variants of polynucleotides and polypeptides may be made by mutagenesis techniques or by direct synthesis. Also included as variants are polypeptides having one or more post-translational modifications, for instance glycosylation, phosphorylation, methylation, ADP ribosylation and the like. Embodiments include methylation of the N-terminal amino acid, phosphorylations of serines and threonines and modification of C-terminal glycines.

“Allele” refers to one of two or more alternative forms of a gene occurring at a given locus in the genome.

“Polymorphism” refers to a variation in nucleotide sequence (and encoded polypeptide sequence, if relevant) at a given position in the genome within a population.

“Single Nucleotide Polymorphism” (SNP) refers to the occurrence of nucleotide variability at a single nucleotide position in the genome, within a population. An SNP may occur within a gene or within intergenic regions of the genome. SNPs can be assayed using Allele Specific Amplification (ASA). For the process at least 3 primers are required. A common primer is used in reverse complement to the polymorphism being assayed. This common primer can be between 50 and 1500 bps from the polymorphic base. The other two (or more) primers are identical to each other except that the final 3′ base wobbles to match one of the two (or more) alleles that make up the polymorphism. Two (or more) PCR reactions are then conducted on sample DNA, each using the common primer and one of the Allele Specific Primers.

“Splice Variant” as used herein refers to cDNA molecules produced from RNA molecules initially transcribed from the same genomic DNA sequence but which have undergone alternative RNA splicing. Alternative RNA splicing occurs when a primary RNA transcript undergoes splicing, generally for the removal of introns, which results in the production of more than one mRNA molecule each of that may encode different amino acid sequences. The term splice variant also refers to the proteins encoded by the above cDNA molecules.

“Identity” reflects a relationship between two or more polypeptide sequences or two or more polynucleotide sequences, determined by comparing the sequences. In general, identity refers to an exact nucleotide to nucleotide or amino acid to amino acid correspondence of the two polynucleotide or two polypeptide sequences, respectively, over the length of the sequences being compared.

“% Identity”—For sequences where there is not an exact correspondence, a “% identity” may be determined. In general, the two sequences to be compared are aligned to give a maximum correlation between the sequences. This may include inserting “gaps” in either one or both sequences, to enhance the degree of alignment. A % identity may be determined over the whole length of each of the sequences being compared (so-called global alignment), that is particularly suitable for sequences of the same or very similar length, or over shorter, defined lengths (so-called local alignment), that is more suitable for sequences of unequal length.

“Similarity” is a further, more sophisticated measure of the relationship between two polypeptide sequences. In general, “similarity” means a comparison between the amino acids of two polypeptide chains, on a residue by residue basis, taking into account not only exact correspondences between a between pairs of residues, one from each of the sequences being compared (as for identity) but also, where there is not an exact correspondence, whether, on an evolutionary basis, one residue is a likely substitute for the other. This likelihood has an associated “score” from which the “% similarity” of the two sequences can then be determined.

Methods for comparing the identity and similarity of two or more sequences are well known in the art. Thus for instance, programs available in the Wisconsin Sequence Analysis Package, version 9.1 (Devereux J et al, Nucleic Acids Res, 12, 387-395, 1984, available from Genetics Computer Group, Madison, Wis., USA), for example the programs BESTFIT and GAP, may be used to determine the % identity between two polynucleotides and the % identity and the % similarity between two polypeptide sequences. BESTFIT uses the “local homology” algorithm of Smith and Waterman (J Mol Biol, 147,195-197, 1981, Advances in Applied Mathematics, 2, 482-489, 1981) and finds the best single region of similarity between two sequences. BESTFIT is more suited to comparing two polynucleotide or two polypeptide sequences that are dissimilar in length, the program assuming that the shorter sequence represents a portion of the longer. In comparison, GAP aligns two sequences, finding a “maximum similarity,” according to the algorithm of Neddleman and Wunsch (J Mol Biol, 48, 443-453, 1970). GAP is more suited to comparing sequences that are approximately the same length and an alignment is expected over the entire length. Preferably, the parameters “Gap Weight” and “Length Weight” used in each program are 50 and 3, for polynucleotide sequences and 12 and 4 for polypeptide sequences, respectively. Preferably, % identities and similarities are determined when the two sequences being compared are optimally aligned.

Other programs for determining identity and/or similarity between sequences are also known in the art, for instance the BLAST family of programs (Altschul S F et al, J Mol Biol, 215, 403-410, 1990, Altschul S F et al, Nucleic Acids Res., 25:389-3402, 1997, available from the National Center for Biotechnology Information (NCBI), Bethesda, Md., USA and accessible through the home page of the NCBI at www.ncbi.nlm.nih.gov) and FASTA (Pearson W R, Methods in Enzymology, 183, 63-99, 1990; Pearson W R and Lipman D J, Proc Nat Acad Sci USA, 85, 2444-2448,1988, available as part of the Wisconsin Sequence Analysis Package).

Preferably, the BLOSUM62 amino acid substitution matrix (Henikoff S and Henikoff J G, Proc. Nat. Acad Sci. USA, 89, 10915-10919, 1992) is used in polypeptide sequence comparisons including where nucleotide sequences are first translated into amino acid sequences before comparison.

Preferably, the program BESTFIT is used to determine the % identity of a query polynucleotide or a polypeptide sequence with respect to a reference polynucleotide or a polypeptide sequence, the query and the reference sequence being optimally aligned and the parameters of the program set at the default value, as hereinbefore described.

“Identity Index” is a measure of sequence relatedness which may be used to compare a candidate sequence (polynucleotide or polypeptide) and a reference sequence. Thus, for instance, a candidate polynucleotide sequence having, for example, an Identity Index of 0.95 compared to a reference polynucleotide sequence is identical to the reference sequence except that the candidate polynucleotide sequence may include on average up to five differences per each 100 nucleotides of the reference sequence. Such differences are selected from the group consisting of at least one nucleotide deletion, substitution, including transition and transversion, or insertion. These differences may occur at the 5′ or 3′ terminal positions of the reference polynucleotide sequence or anywhere between these terminal positions, interspersed either individually among the nucleotides in the reference sequence or in one or more contiguous groups within the reference sequence. In other words, to obtain a polynucleotide sequence having an Identity Index of 0.95 compared to a reference polynucleotide sequence, an average of up to 5 in every 100 of the nucleotides of the in the reference sequence may be deleted, substituted or inserted, or any combination thereof, as hereinbefore described. The same applies mutatis mutandis for other values of the Identity Index, for instance 0.96, 0.97, 0.98 and 0.99.

Similarly, for a polypeptide, a candidate polypeptide sequence having, for example, an Identity Index of 0.95 compared to a reference polypeptide sequence is identical to the reference sequence except that the polypeptide sequence may include an average of up to five differences per each 100 amino acids of the reference sequence. Such differences are selected from the group consisting of at least one amino acid deletion, substitution, including conservative and non-conservative substitution, or insertion. These differences may occur at the amino- or carboxy-terminal positions of the reference polypeptide sequence or anywhere between these terminal positions, interspersed either individually among the amino acids in the reference sequence or in one or more contiguous groups within the reference sequence. In other words, to obtain a polypeptide sequence having an Identity Index of 0.95 compared to a reference polypeptide sequence, an average of up to 5 in every 100 of the amino acids in the reference sequence may be deleted, substituted or inserted, or any combination thereof, as hereinbefore described. The same applies mutatis mutandis for other values of the Identity Index, for instance 0.96, 0.97, 0.98 and 0.99.

The relationship between the number of nucleotide or amino acid differences and the Identity Index may be expressed in the following equation:

nₐ≦xₐ−(xₐ·I),

in which:

nₐ is the number of nucleotide or amino acid differences,

xₐ is the total number of nucleotides or amino acids in SEQ ID NO:1 or SEQ ID NO:2, respectively,

I is the Identity Index,

· is the symbol for the multiplication operator, and

in which any non-integer product of xₐ and I is rounded down to the nearest integer prior to subtracting it from xₐ.

“Homolog” is a generic term used in the art to indicate a polynucleotide or polypeptide sequence possessing a high degree of sequence relatedness to a reference sequence. Such relatedness may be quantified by determining the degree of identity and/or similarity between the two sequences as hereinbefore defined. Falling within this generic term are the terms “ortholog,” and “paralog.” “Ortholog” refers to a polynucleotide or polypeptide that is the functional equivalent of the polynucleotide or polypeptide in another species. “Paralog” refers to a polynucleotide or polypeptide that within the same species which is functionally similar.

“Fusion protein” refers to a protein encoded by two, often unrelated, fused genes or fragments thereof. In one example, EP-A-0 464 533-A discloses fusion proteins comprising various portions of constant region of immunoglobulin molecules together with another human protein or part thereof. In many cases, employing an immunoglobulin Fc region as a part of a fusion protein is advantageous for use in therapy and diagnosis resulting in, for example, improved pharmacokinetic properties [see, e.g., EP-A 0232 262]. On the other hand, for some uses it would be desirable to be able to delete the Fc part after the fusion protein has been expressed, detected and purified.

A histamine-like compound refers to a derivative of histamine (1H-imidazole-4-ethanamine), and in preferred embodiments, it is a compound of formula (I), (II), (III) or (IV):

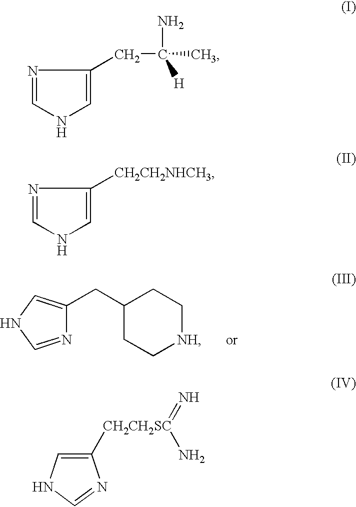

All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

### EXAMPLES

### Example 1

Mammalian Cell Expression

The receptors of the present invention are expressed in either human embryonic kidney 293 (HEK293) cells or adherent dhfr CHO cells. To maximize receptor expression, typically all 5′ and 3′ untranslated regions (UTRs) are removed from the receptor cDNA prior to insertion into a pCDN or pCDNA3 vector. The cells are transfected with individual receptor cDNAs by lipofectin and selected in the presence of 400 mg/ml G418. After 3 weeks of selection, individual clones are picked and expanded for further analysis. HEK293 or CHO cells transfected with the vector alone serve as negative controls. To isolate cell lines stably expressing the individual receptors, about 24 clones are typically selected and analyzed by Northern blot analysis. Receptor mRNAs are generally detectable in about 50% of the G418-resistant clones analyzed.

### Example 2

Ligand Bank for Binding and Functional Assays

A bank of over 600 putative receptor ligands has been assembled for screening. The bank comprises: transmitters, hormones and chemokines known to act via a human seven transmembrane (7TM) receptor; naturally occurring compounds which may be putative agonists for a human 7TM receptor, non-mammalian, biologically active peptides for which a mammalian counterpart has not yet been identified; and compounds not found in nature, but which activate 7TM receptors with unknown natural ligands. This bank is used to initially screen the receptor for known ligands, using both functional (i.e. calcium, cAMP, microphysiometer, oocyte electrophysiology, etc, see below) as well as binding assays.

### Example 3

Ligand Binding Assays

Ligand binding assays provide a direct method for ascertaining receptor pharmacology and are adaptable to a high throughput format. The purified ligand for a receptor is radiolabeled to high specific activity (50-2000 Ci/mmol) for binding studies. A determination is then made that the process of radiolabeling does not diminish the activity of the ligand towards its receptor. Assay conditions for buffers, ions, pH and other modulators such as nucleotides are optimized to establish a workable signal to noise ratio for both membrane and whole cell receptor sources. For these assays, specific receptor binding is defined as total associated radioactivity minus the radioactivity measured in the presence of an excess of unlabeled competing ligand. Where possible, more than one competing ligand is used to define residual nonspecific binding.

### Example 4

Functional Assay in Xenopus Oocytes

Capped RNA transcripts from linearized plasmid templates encoding the receptor cDNAs of the invention are synthesized in vitro with RNA polymerases in accordance with standard procedures. In vitro transcripts are suspended in water at a final concentration of 0.2 mg/ml. Ovarian lobes are removed from adult female toads, Stage V defolliculated oocytes are obtained, and RNA transcripts (10 ng/oocyte) are injected in a 50 nl bolus using a microinjection apparatus. Two electrode voltage clamps are used to measure the currents from individual Xenopus oocytes in response to agonist exposure. Recordings are made in Ca2+ free Barth's medium at room temperature. The Xenopus system can be used to screen known ligands and tissue/cell extracts for activating ligands.

### Example 5

Microphysiometric Assays

Activation of a wide variety of secondary messenger systems results in extrusion of small amounts of acid from a cell. The acid formed is largely as a result of the increased metabolic activity required to fuel the intracellular signaling process. The pH changes in the media surrounding the cell are very small but are detectable by the CYTOSENSOR microphysiometer (Molecular Devices Ltd., Menlo Park, Calif.). The CYTOSENSOR is thus capable of detecting the activation of a receptor which is coupled to an energy utilizing intracellular signaling pathway such as the G-protein coupled receptor of the present invention.

### Example 6

Extract/Cell Supernatant Screening

A large number of mammalian receptors exist for which there remains, as yet, no cognate activating ligand (agonist). Thus, active ligands for these receptors may not be included within the ligand banks as identified to date. Accordingly, the 7TM receptor of the invention is also functionally screened (using calcium, cAMP, microphysiometer, oocyte electrophysiology, etc., functional screens) against tissue extracts to identify natural ligands. Extracts that produce positive functional responses can be sequentially subfractionated until an activating ligand is isolated and identified.

### Example 7

Calcium and cAMP Functional Assays

7TM receptors which are expressed in HEK 293 cells have been shown to be coupled functionally to activation of PLC and calcium mobilization and/or cAMP stimulation or inhibition. Basal calcium levels in the HEK 293 cells in receptor-transfected or vector control cells were observed to be in the normal, 100 nM to 200 nM, range. HEK 293 cells expressing recombinant receptors are loaded with fura 2 and in a single day>150 selected ligands or tissue/cell extracts are evaluated for agonist induced calcium mobilization. Similarly, HEK 293 cells expressing recombinant receptors are evaluated for the stimulation or inhibition of cAMP production using standard cAMP quantitation assays. Agonists presenting a calcium transient or cAMP fluctuation are tested in vector control cells to determine if the response is unique to the transfected cells expressing receptor.

### Example 8

One preferred screening method involves co-transfecting HEK-293 cells with a mammalian expression plasmid encoding a G-protein coupled receptor (GPCR), such as AXOR35, along with a mixture comprised of mammalian expression plasmids cDNAs encoding Gα15 (Wilkie T. M. et al Proc Natl Acad Sci USA 1991 88:10049-10053), Gα16 (Amatruda T. T. et al Proc Natl Acad Sci USA 1991 8:5587-5591, and three chimeric G-proteins referred to as Gqi5, Gqs5, and Gqo5 (Conklin B R et al Nature 1993 363:274-276, Conklin B. R. et al Mol Pharmacol 1996 50:885-890). Following a 24 h incubation the transfected HEK-293 cells are plated into poly-D-lysine coated 96 well black/clear plates (Becton Dickinson, Bedford, Mass.). The cells are assayed on FLIPR (Fluorescent Imaging Plate Reader, Molecular Devices, Sunnyvale, Calif.) for a calcium mobilization response following addition of test ligands. Upon identification of a ligand which stimulates calcium mobilization in HEK-293 cells expressing a given GPCR and the G-protein mixtures, subsequent experiments are performed to determine which, if any, G-protein is required for the functional response. HEK-293 cells are then transfected with the test GPCR, or co-transfected with the test GPCR and Gα15, Gα16, Gqi5, Gqs5, or Gqo5. If the GPCR requires the presence of one of the G-proteins for functional expression in HEK-293 cells, all subsequent experiments are performed with HEK-293 cell co-transfected with the GPCR and the G-protein which gives the best response. Alternatively, the receptor can be expressed in a different cell line, for example RBL-2H3, without additional G-proteins.

By using an analogous method, we found that histamine and a histamine-like compound are ligands for AXOR35. Further we discovered that Gα15 (Feild J A, Foley J J, Testa T T , Nuthulaganti P, Ellis C, Sarau H M, Ames R S. Cloning and characterization of a rabbit ortholog of human Galpha16 and mouse G(alpha)15. FEBS Lett. 1999 October 22;460(1):53-6.)), Gα16, Gqi5, and Gqo5, but not Gqs5, couple to AXOR35.

### Example 9

Prior to challenge with histamine, cells transiently expressing AXOR35 are treated with increasing concentrations of test compound. If the test compound is an agonist, a concentration-dependent fluorescent signal, resulting from intracellular mobilization of calcium, will be observed. This is confirmed if a reduction in signal relative to control cells challenged with histamine (EC80=100 nM) is also observed. This results from desensization of the receptor by the test compound. Agonist activity is as calculated as the concentration of test compound required to produce 50% of a maximum histamine response (EC50). If the test compound is an antagonist, no fluorescent signal from the compound will be produced, but a concentration-dependent reduction in the fluorescent signal of the histamine challenge will be observed. Antagonist activity is calculated as the concentration of test compound required to inhibit 50% of a maximum histamine response (IC50).

### Example 10

Beta-Phenylhistamine, Dihydrochloride (VII)

To a solution of 2-(1H-imidazol-4(5)-yl)-2-phenylacetonitrile (250 mg) (U.S. Pat. No. 5,171,744) in dry THF was added a 0.6 M THF solution of AlH₃ (Brown & Yoon, J. Am. Chem. Soc. 1966, 88, 1464). The resulting solution was stirred at room temperature for 1 h, reflux for 3 h, and then room temperature overnight. A few drops of water were added, and the THF was removed in vacuo. The remaining solid was partially dissolved in water and filtered. The filtrate was extracted with chloroform, and the aqueous layers were concentrated to dryness and precipitated with acetone, providing the crude product. This precipitate was dissolved in water, filtered, basified to pH 11 with K₂CO₃, and extracted with chloroform. The organics were dried and concentrated, providing a green oil, which was treated with ethanolic HCl, precipitating a white solid. The solid was recrystallized from ethanol to provide beta-phenylhistamine, dihydrochloride as white crystals, mp 264-266° C.

### Example 11

beta-(4-Chlorobenzyl)histamine, dihydrochloride (VIII)

1a. Sodium hydroxide (1.6 g) in water (10 mL) was added to a mixture of 4-imidazoleacetonitrile (8 g) and p-chlorobenzaldehyde (15.8 g) in methanol (80 mL). The dark red mixture was left to stand at room temperature for 18 h, and then was heated at reflux for 2 h. The mixture was concentrated in vacuo to approximately 40 mL and was diluted with water (200 mL), resulting in the precipitation of a yellow solid. Collection of the solid by filtration provided the impure product. Recrystallization of the solid using hot ethyl acetate and isolation of three crops of brown crystals provided pure 2-(4-imidazolyl)-p-chlorocinnamontrile, 10.5 g.

1b. Sodium amalgam (150 g, 3%) was added in small portions to a stirring solution of the above 2-(4-imidazolyl)-p-chlorocinnamonitrile (10.5 g) in methanol (500 mL) and water (190 mL) at room temperature. Carbon dioxide was bubbled through the solution to maintain the pH around 9. The resulting mixture was stirred for 1.5 h at room temperature, then heated to a reflux. Another portion of sodium amalgam (50 g) was added, and reflux was continued for 1.5 h. After cooling, the reaction mixture was filtered through Celite, and the filtrate was concentrated in vacuo, leaving a white solid. The solid was extracted twice using boiling 50% ethyl acetate/isopropanol (2×200 mL), and the extracts were concentrated to approximately 80 mL. Leaving the concentrated extracts in the freezer overnight resulted in crystallization of the product, providing the pure 3-p-chlorophenyl-2-(4-imidazolyl)propionitrile.

1c. Aluminum chloride (1.1 g) was added in small portions to dry THF (30 mL) under nitrogen (caution-this produces a violent reaction). Then LiAlH₄ (0.9 g) was added portionwise, and the resulting mixture was stirred for 1.5 h. After this time, the mixture was filtered though Celite, providing a colorless filtrate. The above 3-p-chlorophenyl-2-(4-imidazolyl)propionitrile (2.5 g) was added portionwise to filtrate with stirring, resulting in vigorous bubbling. Once the addition was complete, the reaction mixture was stirred for 30 min at room temperature. Excess hydride was destroyed by the addition of water (dropwise with care!), producing a white precipitate. The mixture was filtered through Celite, washing with ethyl acetate. Concentration of the filtrate and washings provided a thick yellow oil. The oil was dissolved in ethanolic HCl and concentrated to provide a yellow oil. Trituration with isopropanol produced a white solid which was recrystallized from methanol/isopropanol, providing beta-(4-chlorobenzyl)histamine, dihydrochloride as white crystals, mp 254-256° C.

### Example 12

N-(2,5-Difluoro-benzyl)-N′-[2-(3,4-dimethoxy-phenyl)-ethyl]-N″-[2-(1 H-imidazol4-yl)-ethyl]-[1,3,5]triazine-2,4,6-triamine (XIII)

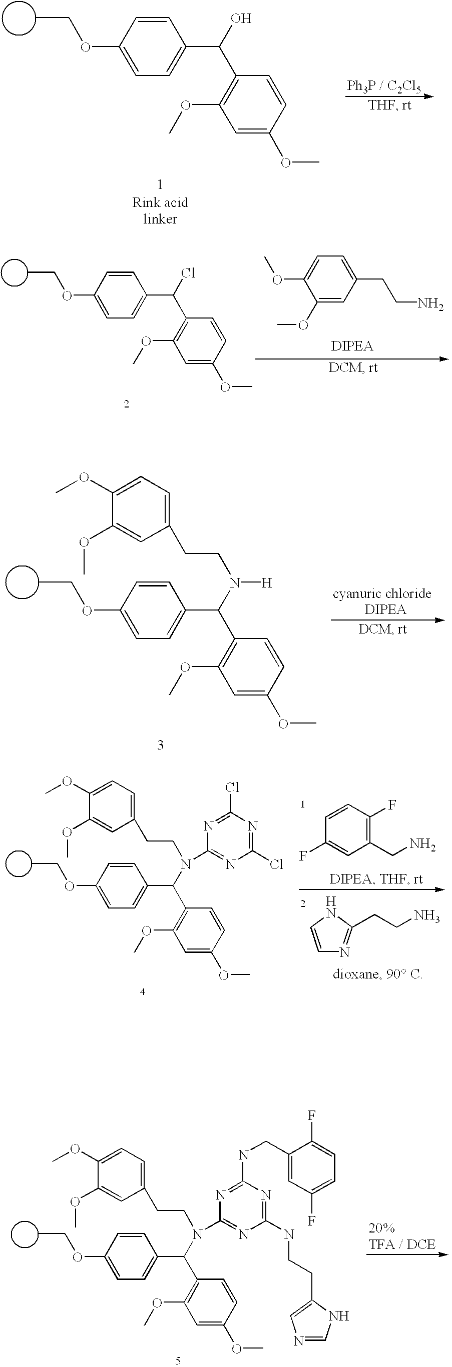

Rink chloride resin 2 was prepared in bulk from commercially-available Rink acid resin 1 (1% crosslinked DVB, polystyrene, 1.37 mmol/g) using the method of Garigipati (Garigipati R. S. Tetrahedron Letter 1997, 38(39), 6807-6810).

To a suspension of Rink chloride resin 2 (25 mg,˜0.034 mmol, 1 equiv) in 0.85 mL dichloromethane was added 3,4-dimethoxyphenethylamine (10 equiv) and diisopropylethylamine (5 equiv). The resulting suspension was mixed overnight at room temperature. Resin 3 was then washed with dichloromethane (2×2 mL), dimethylformamide (2×2 mL), methanol (2×2 mL) and dichloromethane (2×2 mL) then dried under vacuum.

To a −10° C. suspension of resin 3 in 0.85 mL dichloromethane was added under nitrogen atmosphere diisopropylethylamine (12 equiv) and cyanuric chloride (6 equiv). The reaction temperature was then warmed to room temperature and the suspension mixed overnight. Excess reagents and solvent were removed and resin 4 subsequently was washed with dichloromethane (2×2 mL), methanol (2×2 mL), dichloromethane (2×2 mL), methanol (2×2 mL), dichloromethane (2×2 mL), methanol (2×2 mL). Resin 4 was then dried in a vacuum oven overnight (30° C.).

2,5-Difluorobenzylamine (6 equiv) was added to a room-temperature suspension of resin 4 in 1.4 mL of tetrahydrofuran. After 6 hours of mixing, excess reagents and solvent were removed. The resin was then washed with tetrahydrofuran (2×2 mL), methanol (2×2 mL), dichloromethane (2×2 mL), methanol (2×2 mL), dichloromethane (2×2 mL) then dried in a vacuum oven overnight (30° C.).

Histamine (9 equiv) was then added under atmosphere of nitrogen to a suspension of resin in dry dioxane (1.2 mL). The mixture was then heated at 90° C. for 16 hours. Excess reagent and solvent were then removed. Resin 5 was washed with dioxane (2×2 mL), dimethylformamide (2×2 mL), tetrahydrofuran (2×2 mL), methanol (2×2 mL), dichloromethane (2×2 mL), methanol (2×2 mL), dichloromethane (2×2 mL) then dried in a vacuum oven overnight (30° C.).

Resin 5 was then treated 20% trifluoroacetic acid/dichloroethane (2×1.5 mLs, 30 min per treatment). The filtrate was collected into tared 4 mL glass vials then evaporated under reduced pressure in a centrifugal evaporator. The crude product (12 mg) was dissolved in 600 μL of dimethylsulfoxide then subsequently purified by automated preparative HPLC (YMC C18 Reverse Phase, 20×50 mm; 10-95% ACN/water 5 min gradient, 25 mLs/min flow rate). Following evaporation of solvents, pure N-(2,5-Difluoro-benzyl)-N′-[2-(3,4-dimethoxy-phenyl)-ethyl]-N″-[2-(1H-imidazol-4-yl)-ethyl]-[1,3,5]triazine-2,4,6-triamine (XIII) was obtained as the TFA salt (1.2 mg, LCMS (M+H 511.2, UV detection at 214 nm)).

### Example 13

The following Arachindonic Acid-Induced Ear Inflammation and PMA-Induced Ear Inflammation Models can be used to assess the ability of agonists and antagonists of AXOR 35 to affect basic inflammatory response.

Arachidonic Acid-Induced Ear Inflammation

Arachidonic Acid (AA, Sigma Chemicals, St. Louis, Mo.) in acetone (2 mg/20 μL) is applied epicutaneously to the left ear. The thickness of both ears is measured with a constant pressure thickness gauge (Mitutoyo, Japan) 1 hour after treatment and the data is expressed as the change in thickness (mean±standard error of the mean. SEM,×10-3 cm) between treated and untreated ears. Test compounds are administered topically in dimethylacetimide immediately prior to the administration of AA. The treated ears are excised and frozen until assayed for Myeloperoxidase and cytokine levels.

PMA-Induced Ear Inflammation

PMA (Sigma Chemicals, St. Louis, Mo.) in acetone (4 μg/20 μL) is applied epicutaneously to the left ear. The thickness of both ears is measured with a constant pressure thickness gauge (Mitutoyo, Japan) 4 hours after treatment and the data is expressed as the change in thickness (mean±standard error of the mean, SEM,×10-3 cm) between treated and untreated ears. Test compounds are administered topically in dimethylacetimide immediately prior to the administration of PMA. The treated ears are excised and frozen until assayed for Myeloperoxidase and cytokine levels.

### Example 14

The following Oxazalone-induced Ear Inflammation Model can be used to test the effect of agonists and antagonists of AXOR 35 on cellular immunity or delayed type hypersensitivity response.

Oxazalone-Induced Ear Inflammation

The left ear of male BALB/c mice are treated epicutaneously with 1.6% of the contact sensitizing agent, oxazalone. Seven days later, mice are rechallenged with 0.8% of oxazalone and various endpoints are determined 8 hours later. The endpoints include but are not limited to change in ear thickness (mean±SEM,×10-3 cm), Myeloperoxidase content and cytokine levels. Test compounds are administered at least 3 hours prior to the sensitization with the 1.6% of oxazalone.

### Example 15

The following Antigen-Induced Airway Responses In Guinea Pigs models can be used to assess the efficacy of agonists or antagonists of AXOR35 to treat asthma.

Sensitization Protocol

Male Hartley guinea pigs (200-250 g ) are sensitized by i.m. injections of 0.35 ml of a 5% (w/v) ovalbumin (OA)/saline solution into each thigh (0.7 ml total) on days 1 and 4. Guinea pigs are available for use after 4 weeks (conscious experiments) or 6 weeks (anesthetized experiments).

Antigen-Induced Bronchoconstriction in Anesthetized Guinea Pigs

Antigen-induced bronchoconstriction is elicited by i.v. administration of 0.1 mg/kg OA. At the peak of the initial response to OA, a subsequent dose of 0.2 mg/kg OA (0.3 mg/kg, cummulative) is administered. At the peak of the antigen response, 1 cc/kg of a saturated KCl solution is injected. Responses to OA are expressed as a percent of KCl-induced maximal bronchoconstriction. For testing of agents which may affect this response, compounds are administered i.v. (10 min prior to OA challenge) or subcutaneously, intraperitoneal or orally (30 min-2 hr prior to OA challenge).

Aerosol Antigen-Induced Bronchoconstriction and Eosinophil Influx in Conscious Animals

OA-sensitized guinea pigs are placed into a double-flow body plethysmograph (Penn-Century, Philadelphia, Pa.) connected to a Noninvasive Respiratory Analyzer (Buxco Electronics, Sharon, CN) which calculated specific airway conductance (sGaw).

Depending on the experiment, animals may be pretreated with chlorpheniramine (0.1 mg/kg s.c.) to prevent anaphylaxis. For testing of agents which may affect this response, compounds are administered subcutaneously, intraperitoneal or orally (30 min-2 hr prior to OA challenge).

After a 10 min stabilization, an aerosol of 1% OA is generated by a DeVilbiss Pulmosonic nebulizer and delivered for 10 sec at a rate of 250 ml/min via a nosecone built into the plethysmograph.

Results are calculated as percent change in sGaw from baseline readings taken just prior to antigen challenge and reported every 1 to 2 minutes until 10 min post challenge.

Bronchoalveolar lavages (BALs) are performed 24 hours after OA exposure. Guinea pigs are euthanized by pentobarbital overdose and exsanguinated. The lungs are lavaged with 50 ml of DulBecco's PBS (5×10 cc), which is aspirated after a gentle chest massage. The BAL fluid is spun down and the pellet is resuspended in 0.25% NaCl to lyse residual erythrocytes. After centrifugation, the pellet is resuspended again in 0.9% NaCl. After a total cell count, slides are prepared and stained. The cells are identified as eosinophils, neutrophils and mononuclear cells by counting, a minimum of 200 cells and expressing the results as percent of total cells.

| | | |
|---|---|---|
| ATGCCAGATACTAATAGCACAATCAATTTATCACTAAGCA | 40 | SEQ ID NO: 1 |
| CTCGTGTTACTTTAGCATTTTTTATGTCCTTAGTAGCTTT | 80 | |
| TGCTATAATGCTAGGAAATGCTTTGGTCATTTTAGCTTTT | 120 | |
| GTGGTGGACAAAAACCTTAGACATCGAAGTAGTTATTTTT | 160 | |
| TTCTTAACTTGGCCATCTCTGACTTCTTTGTGGGTGTGAT | 200 | |
| CTCCATTCCTTTGTACATCCCTCACACGCTGTTCGAATGG | 240 | |
| GATTTTGGAAAGGAAATCTGTGTATTTTGGCTCACTACTG | 280 | |
| ACTATCTGTTATGTACAGCATCTGTATATAACATTGTCCT | 320 | |
| CATCAGCTATGATCGATACCTGTCAGTCTCAAATGCTGTG | 360 | |
| TCTTATAGAACTCAACATACTGGGGTCTTGAAGATTGTTA | 400 | |
| CTCTGATGGTGGCCGTTTGGGTGCTGGCCTTCTTAGTGAA | 440 | |
| TGGGCCAATGATTCTAGTTTCAGAGTCTTGGAAGGATGAA | 480 | |
| GGTAGTGAATGTGAACCTGGATTTTTTTCGGAATGGTACA | 520 | |
| TCCTTGCCATCACATCATTCTTGGAATTCGTGATCCCAGT | 560 | |
| CATCTTAGTCGCTTATTTCAACATGAATATTTATTGGAGC | 600 | |
| CTGTGGAAGCGTGATCATCTCAGTAGGTGCCAAAGCCATC | 640 | |
| CTGGACTGACTGCTGTCTCTTCCAACATCTGTGGACACTC | 680 | |
| ATTCAGAGGTAGACTATCTTCAAGGAGATCTCTTTCTGCA | 720 | |
| TCGACAGAAGTTCCTGCATCCTTTCATTCAGAGAGACAGA | 760 | |
| GGAGAAAGAGTAGTCTCATGTTTTCCTCAAGAACCAAGAT | 800 | |
| GAATAGCAATACAATTGCTTCCAAAATGGGTTCCTTCTCC | 840 | |
| CAATCAGATTCTGTAGCTCTTCACCAAAGGGAACATGTTG | 880 | |
| AACTGCTTAGAGCCAGGAGATTAGCCAAGTCACTGGCCAT | 920 | |
| TCTCTTAGGGGTTTTTGCTGTTTGCTGGGCTCCATATTCT | 960 | |
| CTGTTCACAATTGTCCTTTCATTTTATTCCTCAGCAACAG | 1000 | |
| GTCCTAAATCAGTTTGGTATAGAATTGCATTTTGGCTTCA | 1040 | |
| GTGGTTCAATTCCTTTGTCAATCCTCTTTTGTATCCATTG | 1080 | |
| TGTCACAAGCGCTTTCAAAAGGCTTTCTTGAAAATATTTT | 1120 | |
| GTATAAAAAAGCAACCTCTACCATCACAACACAGTCGGTC | 1160 | |
| AGTATCTTCTTAA | 1173 | |
| MPDTNSTINLSLSTRVTLAFFMSLVAFAIMLGNALVILAF | 40 | SEQ ID NO: 2 |
| VVDKNLRHRSSYFFLNLAISDFFVGVISIPLYIPHTLFEW | 80 | |
| DFGKEICVFWLTTDYLLCTASVYNIVLISYDRYLSVSNAV | 120 | |
| SYRTQHTGVLKIVTLMVAVWVLAFLVNGPMILVSESWKDE | 160 | |
| GSECEPGFFSEWYILAITSFLEFVIPVILVAYFNMNIYWS | 200 | |
| LWKRDHLSRCQSHPGLTAVSSNICGHSFRGRLSSRRSLSA | 240 | |
| STEVPASFHSERQRRKSSLMFSSRTKMNSNTIASKMGSFS | 280 | |
| QSDSVALHQREHVELLRARRLAKSLAILLGVFAVCWAPYS | 320 | |
| LFTIVLSFYSSATGPKSVWYRIAFWLQWFNSFVNPLLYPL | 360 | |

| | |
|---|---|
| CHKRFQKAFLKIFCIKKQPLPSQHSRSVSS | 390 |

What is claimed is:

1. An isolated polypeptide selected from the group consisting of:
(a) an isolated polypeptide encoded by a polynucleotide comprising the sequence of SEQ ID NO:1;
(b) an isolated polypeptide comprising a polypeptide sequence having at least 95% identity to the polypeptide sequence of SEQ ID NO:2;
(c) an isolated polypeptide comprising the polypeptide sequence of SEQ ID NO:2;
(d) an isolated polypeptide having at least 95% identity to the polypeptide sequence of SEQ ID NO:2;
(e) the polypeptide sequence of SEQ ID NO:2; and
(f) fragments and variants of such polypeptides in (a) to (e).

2. An isolated polynucleotide selected from the group consisting of:
(a) an isolated polynucleotide comprising a polynucleotide sequence having at least 95% identity to the polynucleotide sequence of SEQ ID NO:1;
(b) an isolated polynucleotide comprising the polynucleotide of SEQ ID NO:1;
(c) an isolated polynucleotide having at least 95% identity to the polynucleotide of SEQ ID NO:1;
(d) the isolated polynucleotide of SEQ ID NO:1;
(e) an isolated polynucleotide comprising a polynucleotide sequence encoding a polypeptide sequence having at least 95% identity to the polypeptide sequence of SEQ ID NO:2;
(f) an isolated polynucleotide comprising a polynucleotide sequence encoding the polypeptide of SEQ ID NO:2;
(g) an isolated polynucleotide having a polynucleotide sequence encoding a polypeptide sequence having at least 95% identity to the polypeptide sequence of SEQ ID NO:2;
(h) an isolated polynucleotide encoding the polypeptide of SEQ ID NO:2;
(i) an isolated polynucleotide with a nucleotide sequence of at least 100 nucleotides obtained by screening a library under stringent hybridization conditions with a labeled probe having the sequence of SEQ ID NO: 1 or a fragment thereof having at least 15 nucleotides; and
(j) a polynucleotide which is the RNA equivalent of a polynucleotide of (a) to (i); or a polynucleotide sequence complementary to said isolated polynucleotide and polynucleotides that are variants and fragments of the above mentioned polynucleotides or that are complementary to above mentioned polynucleotides, over the entire length thereof.

3. An antibody immunospecific for the polypeptide of claim 1.

4. An antibody as claimed in claim 3 which is a polyclonal antibody.

5. An expression vector comprising a polynucleotide capable of producing a polypeptide of claim 1 when said expression vector is present in a compatible host cell.

6. A process for producing a recombinant host cell which comprises the step of introducing an expression vector comprising a polynucleotide capable of producing a polypeptide of claim 1 into a cell such that the host cell, under appropriate culture conditions, produces said polypeptide.

7. A recombinant host cell produced by the process of claim 6.

8. A membrane of a recombinant host cell of claim 7 expressing said polypeptide.

9. A process for producing a polypeptide which comprises culturing a host cell of claim 7 under conditions sufficient for the production of said polypeptide and recovering said polypeptide from the culture.

10. A method for identifying agonist or antagonist of a AXOR35 polypeptide which comprises:
contacting a cell expressing on the surface thereof the polypeptide, said polypeptide being associated with a second component capable of providing a detectable signal in response to the binding of a compound to said polypeptide, with a compound to be screened under conditions to permit binding to the polypeptide; and
determining whether the compound binds to and activates or inhibits the polypeptide by measuring the level of a signal generated from the interaction of the compound with the polypeptide.

11. A method for identifying agonist or antagonist of a AXOR35 polypeptide which comprises:
(a) contacting a cell expressing on the surface thereof the polypeptide, said polypeptide being associated with a second component capable of providing a detectable signal in response to the binding of a compound to said polypeptide, with a compound to be screened under conditions to permit binding to the polypeptide; and
(b) determining whether the compound binds to and activates or inhibits the polypeptide by comparing the level of a signal generated from the interaction of the compound with the polypeptide with the level of a signal without the presence of the compound.

12. A method of claim 10 or 11 which further comprises conducting the identification of agonist or antagonist in the presence of labeled or unlabeled histamine or a histamine-like compound.

13. A method for identifying agonist or antagonist of a AXOR35 polypeptide which comprises:
determining the inhibition of binding of a ligand to cells which have the polypeptide on the surface thereof, or to cell membranes containing the polypeptide, in the presence of a candidate compound under conditions to permit binding to the polypeptide, and determining the amount of ligand bound to the polypeptide, such that a compound capable of causing reduction of binding of a ligand is an agonist or antagonist.

14. A method of claim 13 in which the ligand is labeled or unlabeled histamine or a histamine-like compound.

15. A method of screening for AXOR35 polypeptide antagonist or agonist comprising the steps of:
(a) incubating a labeled histamine and histamine-like compound with a whole cell expressing AXOR35 polypeptide on the cell surface, or cell membrane containing AXOR35 polypeptide;
(b) measuring the amount of labeled histamine or a histamine-like compound bound to the whole cell or the cell membrane;
(c) adding a candidate compound to a mixture of labeled histamine or a histamine-like compound and the whole cell or the cell membrane of step (a) and allowing to attain equilibrium;
(d) measuring the amount of labeled histamine or a histamine-like compound bound to the whole cell or the cell membrane after step (c); and
(e) comparing the difference in the labeled histamine or a histamine-like compound bound in step (b) and (d), such that the compound which causes the reduction in binding in step (d) is an agonist or antagonist.

16. A method of treating a disease of the invention by administering to a patient in need thereof an agonist or an antagonist identified by any of the methods of claims 10-15.

17. The disease of claim 16 that is asthma.

18. A method of inhibiting or promoting the function of lymphocytes, macrophages, eosinophils, or neutrophils in diseased tissue comprising the step of administering to a patient in need thereof AXOR35 agonists or antagonists.

19. A method of inhibiting or promoting the function of lymphocytes, macrophages, eosinophils, or neutrophils in diseased tissue comprising the step of administering to a patient in need thereof AXOR35 agonists or antagonists identified by any of the methods of claims of 10-15.

20. The disease tissue of claim 18 or 19 that is asthmatic lung.

21. A method of agonizing or antagonizing AXOR35 in order to inhibit or promote the function of lymphocytes, macrophages, eosinophils, or neutrophils in diseased tissue comprising the step of administering to a patient in need thereof AXOR35 agonists or antagonists.

22. A method of agonizing or antagonizing AXOR35 in order to inhibit or promote the function of lymphocytes, macrophages, eosinophils, or neutrophils in diseased tissue comprising the step of administering to a patient in need thereof AXOR35 agonists or antagonists identified by any of the methods of claim 10-15.

23. The disease tissue of claim 21 or 22 that is asthmatic lung.
